(19) United States
(12) Patent Application Publication
(10) Pub. No.: US 2015/0011756 A1
(43) Pub. Date: Jan. 8, 2015

(54) PROCESS FOR PREPARATION OF RIVAROXABAN AND INTERMEDIATES THEREOF

(71) Applicant: MEGAFINE PHARMA (P) LTD, Mumbi, Maharashtra (IN)

(72) Inventors: Vijayavitthal Thippannachar Mathad, Nashik Maharashtra (IN); Nilesh Sudhir Patil, Jalgaon Maharashtra (IN); Navnath Chintaman Niphade, Nashik Maharashtra (IN); Anil Chaturlal Mali, Nashik Maharashtra (IN); Mahendra Bhagirath Bodake, Nashik Maharashtra (IN); Sharad Subhash Ippar, Nashik Maharashtra (IN); Rajesh Talla, Prakasham (Dist.) Andhrapradesh (IN)

(21) Appl. No.: 14/376,966

(22) PCT Filed: Feb. 5, 2013
(86) PCT No.: PCT/IN2013/000077

§ 371 Date: Aug. 6, 2014

(30) Foreign Application Priority Data
Feb. 6, 2012 (IN) 339/MUM/2012

(51) Int. Cl. C07D 413/14; C07D 333/38

(52) U.S. Cl. CPC C07D 413/14; C07D 333/38
USPC 544/137; 549/71

(57) ABSTRACT

An improved process for the preparation of Rivaroxaban wherein the process substantially eliminates the potential impurities. process for preparation of Rivaroxaban which uses a novel intermediate. A process for preparing the novel intermediate which is used for the preparation of Rivaroxaban.

This application claims priority from Indian patent application no. 339/MUM/2012 filed on 6 Feb. 2012.

### FIELD OF THE INVENTION

The present invention relates to a method for the preparation of 5-chloro-N-({(5S)-2-oxo-3-[4-(3-oxomorpholin-4-yl)phenyl]-1,3-oxazolidin-5-yl}methyl)thiophene-2-carboxamide, Rivaroxaban of formula (I), and to new intermediates used for the preparation of Rivaroxaban thereof.

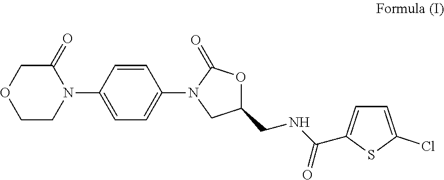 [SMILES: O=C(NC[C@H]1CN(c2ccc(N3CCOCC3=O)cc2)C(=O)O1)c1ccc(Cl)s1]

The present invention also relates to a method for the preparation of Rivaroxaban wherein, the said invention substantially eliminates the impurities formed during the preparation of Rivaroxaban.

### BACKGROUND OF THE INVENTION

5-chloro-N-({(5S)-2-oxo-3-[4-(3-oxomorpholin-4-yl)phenyl]-1,3-oxazolidin-5-yl}methyl)thiophene-2-carboxamide (herein “Rivaroxaban”) also known as Xarelto®, has a CAS number of 366789-02-8, a molecular formula of C₁₉H₁₈ClN₃O₅S and the following structure:

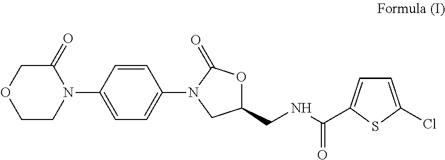 [SMILES: O=C(NC[C@H]1CN(c2ccc(N3CCOCC3=O)cc2)C(=O)O1)c1ccc(Cl)s1]

Rivaroxaban is an orally active direct factor Xa (FXa) inhibitor drug, used for the prevention and treatment of various thromboembolic diseases, in particular pulmonary embolism, deep venous thrombosis, myocardial infarction, angina pectoris, reocclusion and restenosis after angioplasty or aortocoronary bypass, cerebral stroke, transitory ischemic attacks, and peripheral arterial occlusive diseases.

WO 01/47919 application describes a method for preparing Rivaroxaban of formula (I), with an overall yield of 69%, wherein 4-(4-aminophenyl)morpholin-3-one compound of formula (II) is reacted with 2-[(2S)-oxiran-2-ylmethyl]-1H-isoindole-1,3(2H)-dione, compound of formula (III), in presence of solvent namely, ethanol and water mixture to obtain 2-[(2R)-2-hydroxy-3-{[4-(3-oxomorpholin-4-yl)phenyl]amino}propyl]-1H-isoindole-1,3(2H)-dione compound of formula (IV). Subsequently, compound of formula (IV) is converted to 2-({(5S)-2-oxo-3-[4-(3-oxomorpholin-4-yl)phenyl]-1,3-oxazolidin-5-yl}methyl)-1H-isoindole-1,3(2H)-dione of formula (V) with a phosgene equivalent namely, 1,1′-carbonylbis(1H-imidazole). Removal of the pthalamide protecting group from compound of formula (V) affords 4-{4-[(5S)-5-(aminomethyl)-2-oxo-1,3-oxazolidin-3-yl]phenyl}morpholin-3-one of formula (VI), which is then coupled with 5-chlorothiophene-2-carbonyl chloride of formula (VIII) to give 5-chloro-N-({(5S)-2-oxo-3-[4-(3-oxomorpholin-4-yl)phenyl]-1,3-oxazolidin-5-yl}methyl)thiophene-2-carboxamide i.e. Rivaroxaban of formula (I) as shown in scheme-1;

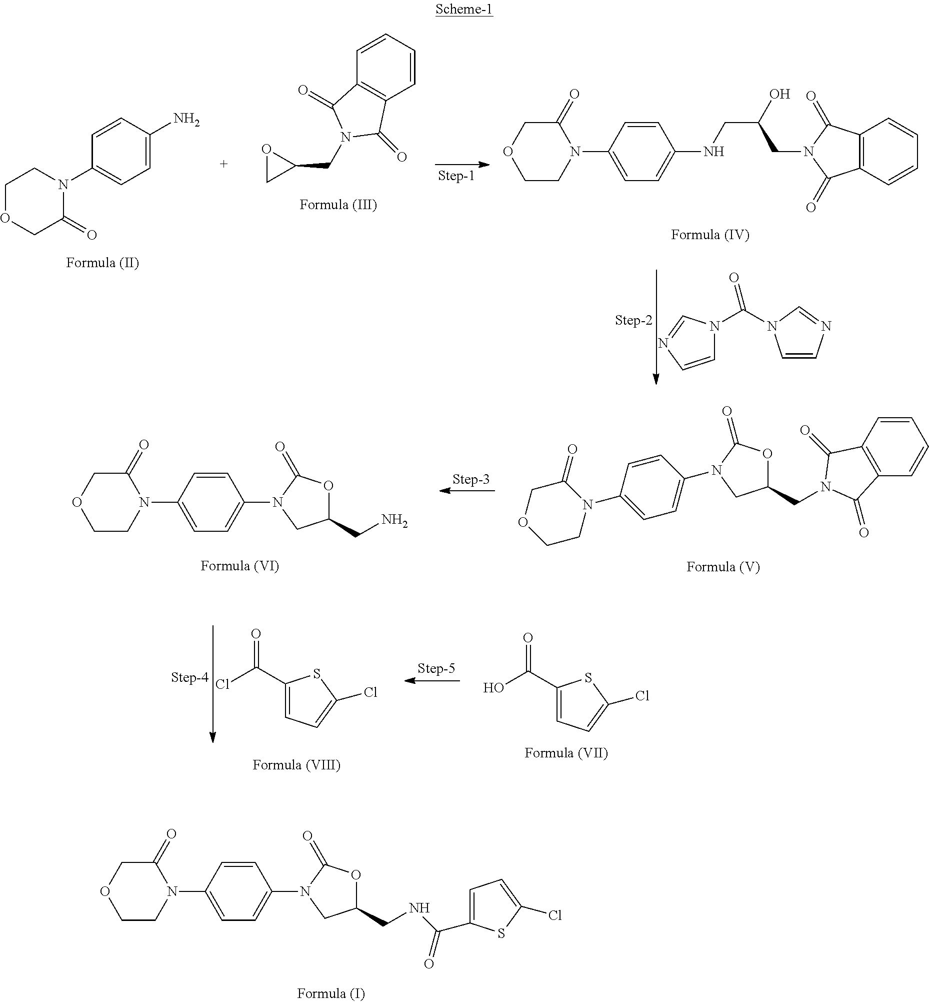

However, we have analyzed the process disclosed in WO 01/47919, which exhibits various disadvantages in the reaction management for the preparation of Rivaroxaban on industrial scale. Some of the limitations are as follows:
process disclosed in WO 01/47919 is lengthy (Step-1 requires 48 hours and Step-2 requires 40 hours) and time consuming;
excess loading (4 mole equivalents) of expensive compound of formula (III) for the reaction between compound of formula (II) with compound of formula (III), makes the whole process expensive and cumbersome as the reaction mass of the first reaction needs to be filtered and the obtained mass was subjected for the same reaction using compound of formula (III);
diethylether employed for washing the compound of formula (IV) is flammable thus not safe
process disclosed in WO 01/47919 involve purification of the intermediate compound of Formula (V) and Rivaroxaban of formula (I) by column chromatography which is industrially not feasible. WO 01/47919 process is unsafe and not eco-friendly due to use of carcinogenic pyridine as a solvent and base

Hence, there is a need for a solution that overcomes the above stated limitations.

The present invention proposes a process for preparation of Rivaroxaban and novel intermediates used for the preparation of Rivaroxaban thereof; which is economic, efficient, eco-friendly, and eliminates extensive laborious work-up.

### OBJECTS OF THE PRESENT INVENTION

The primary object of the present invention is to provide, efficient, economic and industrially feasible process for preparation of Rivaroxaban of formula (I).

Another object of the present invention is to provide novel intermediate for the preparation of Rivaroxaban of formula (I).

Yet another object of the present invention is to provide a process for preparation of Rivaroxaban of formula (I); wherein the said process eliminates laborious workup and extensive purifications. Hence, makes the process simple, easy and user friendly.

Yet another object of the present invention is to provide a process for preparation of Rivaroxaban of formula (I), wherein the obtained Rivaroxaban is substantially free from impurities and thereby eliminating the required purification steps and further making the process cost effective and efficient.

### BRIEF DESCRIPTION OF DRAWINGS

FIG. 1 of the present invention illustrates X-ray powder diffraction (XRD) pattern of hydrochloride salt of compound of formula (VI), prepared according to example 3.

FIG. 2 of the present invention illustrates Infrared spectrum (IR) of hydrochloride salt of compound of formula (VI), prepared according to example 3.

FIG. 3 of the present invention illustrates X-ray powder diffraction (XRD) pattern of compound of formula (VI) as a free base, prepared according to example 6.

FIG. 4 of the present invention illustrates Infrared spectrum (IR) of compound of formula (VI) as a free base, prepared according to example 6.

### DETAILED DESCRIPTION OF THE INVENTION

Before the present invention is described, it is to be understood that this invention is not limited to particular methodologies and materials described, as these may vary as per the person skilled in the art. It is also to be understood that the terminology used in the description is for the purpose of describing the particular embodiments only, and is not intended to limit the scope of the present invention.

Before the present invention is described, it is to be understood that unless defined otherwise, all technical and scientific terms used herein have the same meaning as commonly understood by one of ordinary skill in the art to which this invention belongs. Further, it is to be understood that the present invention is not limited to the methodologies and materials similar or equivalent to those described herein can be used in the practice or testing of the present invention, the preferred methods and materials are described, as these may vary within the specification indicated. Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims. Further the terms disclosed embodiments are merely exemplary methods of the invention, which may be embodied in various forms.

A term herein “reflux temperature” means the temperature at which the solvent or the solvent system refluxes or boils at atmospheric pressure.

The term “substantially free of” in reference to a composition, as used herein, means that an absent substance cannot be detected in the composition by methods known to those skilled in the art at the time of the filing of this application.

In one of the embodiments, the present invention provides an improved process for the preparation of Rivaroxaban of formula (I) comprising:
a) reacting, 4-(4-aminophenyl)morpholine-3-one of formula (II) with 2-[(2S)-oxiran-2-ylmethyl]-1H-isoindole-1,3(2H)-dione of formula (III) in a first solvent to obtain 2-[(2R)-2-hydroxy-3-{[4-(3-oxomorpholin-4-yl)phenyl]amino}propyl]-1H-isoindole-1,3(2H)-dione of formula (IV);

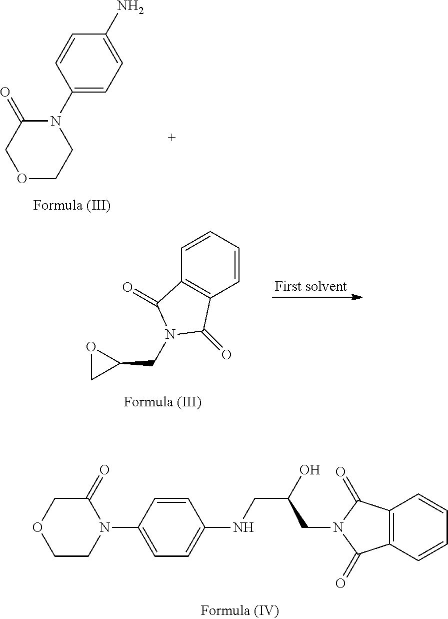 [SMILES: Nc1ccc(N2CCOCC2=O)cc1 ; O=C1c2ccccc2C(=O)N1C[C@H]1CO1 ; O=C1c2ccccc2C(=O)N1C[C@H](O)CNc1ccc(N2CCOCC2=O)cc1]

b) preparing compound of formula (V) by reacting compound of formula (IV) using phosgene or phosgene equivalents or anhydrides or bis(aryl) carbonate of formula (XVI) in a second solvent and optionally in presence of a base,

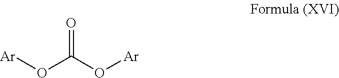

wherein, Ar can be phenyl, substituted phenyl, selected from p-nitrophenyl, 4-(trifluoromethyl)phenyl, 4-cyanophenyl and the like;

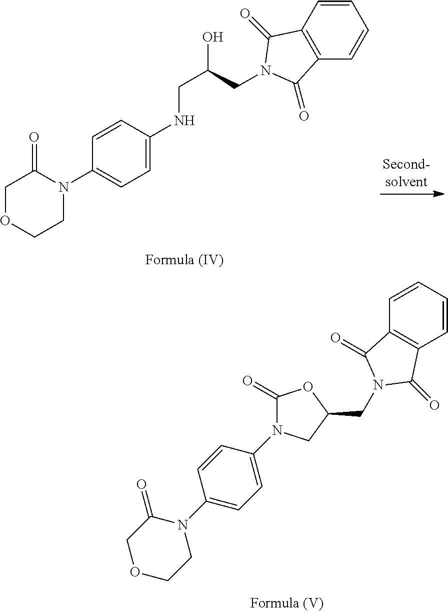 [SMILES: O=C1c2ccccc2C(=O)N1C[C@H](O)CNc1ccc(N2CCOCC2=O)cc1 ; O=C1c2ccccc2C(=O)N1C[C@H]1CN(c2ccc(N3CCOCC3=O)cc2)C(=O)O1]

c) eliminating the pthalamide group from compound of formula (V) in the said first solvent using a de-protecting agent to obtain 4-{4-[(5S)-5-(aminomethyl)-2-oxo-1,3-oxazolidin-3-yl]phenyl}morpholin-3-one formula (VI); optionally isolating and purifying compound of formula (VI); optionally converting to its acid addition salt;

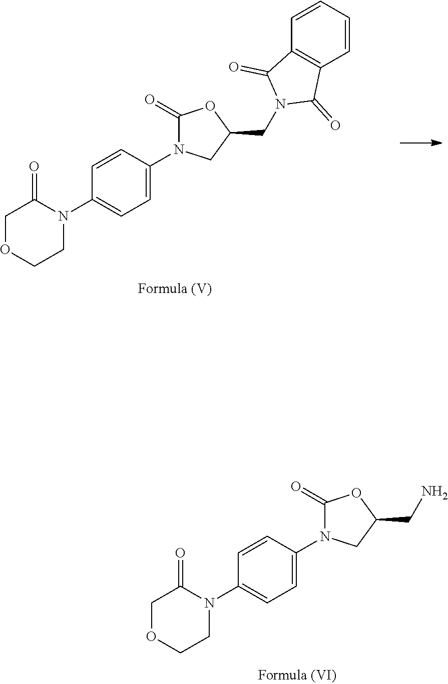 [SMILES: O=C1c2ccccc2C(=O)N1C[C@H]1CN(c2ccc(N3CCOCC3=O)cc2)C(=O)O1 ; NC[C@H]1CN(c2ccc(N3CCOCC3=O)cc2)C(=O)O1]

d) reacting compound of formula (VI) or its acid addition salt with 5-chlorothiophene-2-carbonyl chloride of formula (VIII) in a third solvent and in presence of a base to obtain Rivaroxaban of formula (I), and isolating compound of formula (I);

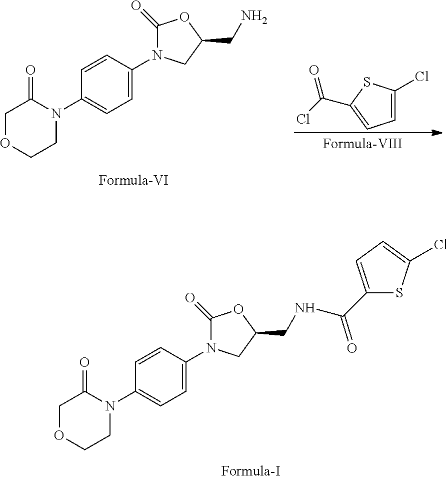 [SMILES: NC[C@H]1CN(c2ccc(N3CCOCC3=O)cc2)C(=O)O1 ; O=C(Cl)c1ccc(Cl)s1 ; O=C(NC[C@H]1CN(c2ccc(N3CCOCC3=O)cc2)C(=O)O1)c1ccc(Cl)s1]

e) optionally, purifying Rivaroxaban of formula (I).

Compound of formula (VI) and Rivaroxaban compound of formula (I) may be optionally converted to its acid addition salt by treating it with suitable acids to obtain either acetate, hydrochloride, maleate, tosylate, formate, tartrate, mesylate, oxalate, fumarate, succinate or the like.

Preferably, compound of formula (VI) is acetate or hydrochloride salt.

According to another embodiment of the present invention, compound of formula (VI) may be further purified either by acid-base treatment, or solvent crystallization, or converting into its acid addition salts.

The acid addition salts of Formula (VI) can be prepared by treating the same with suitable acids; wherein the said acid includes organic and inorganic acids such as but not limited to hydrochloric acid, acetic acid, organic carboxylic acid like tartaric acid, fumaric acid, succinic or oxalic acid.

According to another embodiment, the present invention provides a process for purification of compound of formula (VI), comprising:
i. contacting acid addition salt of compound of formula (VI) in a solvent and in presence of base;
ii. treating reaction mass obtained in step (i) with acid to obtain precipitate of suitable acid addition salt of compound of formula (VI); and
iii. filtering the precipitate, washing the precipitate with solvent and drying to obtain pure acid addition salt of compound of formula (VI).

The acid addition salts of Formula (VI) can be prepared by treating the same with suitable acids; wherein the said acid includes organic and inorganic acids such as but not limited to acetic acid, organic carboxylic acid like tartaric acid, fumaric acid, succinic acid or oxalic acid.

The base used in step (i) is selected from organic or inorganic base.

The organic base is selected from diisopropylamine, 1,8-Diazabicyclo[5.4.0]undec-7-ene, 1,5-Diazabicyclo[4.3.0]non-5-ene, 4-Dimethylaminopyridine, di-isopropylethylamine, triethylamine, and the like

The inorganic base is selected from the group comprising of alkali metal carbonates, alkali metal bicarbonates or alkali metal hydroxides. Preferably, the base used is triethylamine.

The solvent used in step (i) and step (iii) may be either same or different; wherein the said solvent is an organic solvent selected from the group comprising aliphatic hydrocarbons such as but not limited to hexane, cyclohexane, heptane and the like, aromatic hydrocarbons such as but not limited to toluene, xylene and the like; amides such as dialkylformamides such as but not limited to dimethyl formamide and dialkylacetamides such as but not limited to dimethyl acetamide and the like; ethers such as but not limited to di-isopropyl ether, methyl tert. Butyl ether and the like, cyclic ethers such as but not limited to tetrahydrofuran and the like, substituted cyclic ethers such as but not limited to 2-methyl tetrahydrofuran and the like, esters such as but not limited to ethyl acetate, isopropyl acetate and the like, alcohols such as but not limited to methanol, ethanol, isopropyl alcohol, butanol and the like, ketones such as but not limited to acetone, methyl ethyl ketone, Methyl iso-butyl ketone (MIBK) and the like dialkylsulfoxides such as but not limited to dimethyl sulfoxide (DMSO) and the like, nitriles such as but limited to acetonitrile, propionitrile and the like, ionic liquids, halogenated aliphatic hydrocarbons such as but not limited to di-chloromethane, dichloroethane, chloroform, 1,2-dichloroethane and the like and or mixtures thereof.

Preferably, the solvent used in step (i) and (iii) is dichloromethane, methanol or mixtures thereof.

The first solvent used in step (a) and step (c) may be either same or different; wherein the said solvent is an organic solvent selected from the group comprising aliphatic hydrocarbons such as but not limited to hexane, cyclohexane, heptane and the like, aromatic hydrocarbons such as but not limited to toluene, xylene and the like; amides such as dialkylformamides such as but not limited to dimethyl formamide and dialkylacetamides such as but not limited to dimethyl acetamide and the like; ethers such as but not limited to di-isopropyl ether, methyl tert. Butyl ether and the like, cyclic ethers such as but not limited to tetrahydrofuran and the like, substituted cyclic ethers such as but not limited to 2-methyl tetrahydrofuran and the like, esters such as but not limited to ethyl acetate, isopropyl acetate and the like, alcohols such as but not limited to methanol, ethanol, isopropyl alcohol, butanol and the like, ketones such as but not limited to acetone, methyl ethyl ketone, Methyl iso-butyl ketone (MIBK) and the like dialkylsulfoxides such as but not limited to dimethyl sulfoxide (DMSO) and the like, nitriles such as but limited to acetonitrile, propionitrile and the like, ionic liquids, halogenated aliphatic hydrocarbons such as but not limited to di-chloromethane, dichloroethane, chloroform, 1,2-dichloroethane and the like and water or mixtures thereof.

Preferably, the said first solvent used in step (a) may be alcohol, amides, water or mixture thereof. More preferably, the said solvent is isopropyl alcohol and water mixture.

Preferably, the said first solvent used in step (c) may be alcohol, nitriles, formamides, water or mixture thereof. More preferably, the said solvent is methanol.

The step (a) and (c) is carried out at temperature in the range of 25° C. to reflux temperature of the said solvent.

The second solvent used in step (b) for the preparation of compound of formula (V) wherein the said solvent is an organic solvent selected from the group comprising aliphatic hydrocarbons such as but not limited to hexane, cyclohexane, heptane and the like, aromatic hydrocarbons such as but not limited to toluene, xylene and the like; amides such as dialkylformamides such as but not limited to dimethyl formamide and dialkylacetamides such as but not limited to dimethyl acetamide and the like; ethers such as but not limited to di-isopropyl ether, methyl tert. Butyl ether and the like, cyclic ethers such as but not limited to tetrahydrofuran and the like, substituted cyclic ethers such as but not limited to 2-methyl tetrahydrofuran and the like, ketones such as but not limited to acetone, methyl ethyl ketone, Methyl iso-butyl ketone (MIBK) and the like dialkylsulfoxides such as but not limited to dimethyl sulfoxide (DMSO) and the like, esters such as but not limited to ethyl acetate, isopropyl acetate and the like, nitriles such as but limited to acetonitrile, propionitrile and the like, ionic liquids, halogenated aliphatic hydrocarbons such as but not limited to di-chloromethane, dichloroethane, chloroform, 1,2-dichloroethane and the like or mixtures thereof.

Preferably, the said second solvent used in step (b) may be hydrocarbons, ethers or mixtures thereof. More preferably, the said second solvent is hydrocarbons such as halogenated hydrocarbons namely, dichloromethane, dichloroethane and the like.

The third solvent used in step (d) is an organic solvent selected from the group comprising aliphatic hydrocarbons such as but not limited to hexane, cyclohexane, heptane and the like, aromatic hydrocarbons such as but not limited to toluene, xylene and the like; amides such as dialkylformamides such as but not limited to dimethyl formamide and dialkylacetamides such as but not limited to dimethyl acetamide and the like; ethers such as but not limited to di-isopropyl ether, methyl tert. Butyl ether and the like, cyclic ethers such as but not limited to tetrahydrofuran and the like, substituted cyclic ethers such as but not limited to 2-methyl tetrahydrofuran and the like, ketones such as but not limited to acetone, methyl ethyl ketone, Methyl iso-butyl ketone (MIBK) and the like dialkylsulfoxides such as but not limited to dimethyl sulfoxide (DMSO) and the like, esters such as but not limited to ethyl acetate, isopropyl acetate and the like, nitriles such as but limited to acetonitrile, propionitrile and the like, ionic liquids, halogenated aliphatic hydrocarbons such as but not limited to dichloromethane, dichloroethane, chloroform, 1,2-dichloroethane and the like and water or mixtures thereof.

The base used in step (b) and step (d) is selected from organic or inorganic base.

The organic base is selected from diisopropylamine, 1,8-Diazabicyclo[5.4.0]undec-7-ene, 1,5-Diazabicyclo[4.3.0]non-5-ene, 4-Dimethylaminopyridine, di-isopropylethylamine, triethylamine, and the like

The inorganic base is selected from the group comprising of alkali metal carbonates, alkali metal bicarbonates or alkali metal hydroxides.

In a preferred embodiment, the isolation of Rivaroxaban of formula (I) in step (d) may be carried out in a mixture of acid, the third solvent and water.

Optionally, step (d) can be carried out in biphasic medium and optionally in the presence of a phase transfer catalyst.

According to another embodiment, the present invention provides a process for preparation of Rivaroxaban of formula (I);

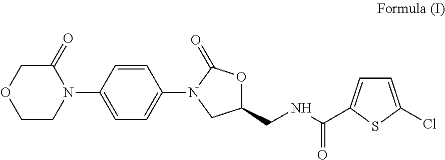 [SMILES: O=C(NC[C@H]1CN(c2ccc(N3CCOCC3=O)cc2)C(=O)O1)c1ccc(Cl)s1]

comprising reacting compound of formula (VI) or its acid addition salt

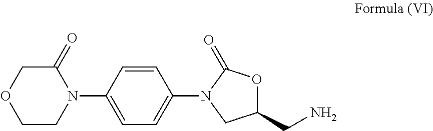 [SMILES: NC[C@H]1CN(c2ccc(N3CCOCC3=O)cc2)C(=O)O1]

with compound of formula (IX)

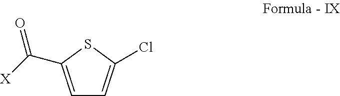

Wherein; X may be sulfonyloxy, imidazole, triazole, tetrazole, alkoxy, substituted alkoxy, tri-halomethoxy, N-hydroxysuccinamide, p-nitrophenol, N-hydroxythalamide, N-hydroxybenzotriazole, or OR;

wherein; R may be alkyl, aryl and substituted aryl;
in solvent and in presence of a base to provide Rivaroxaban of formula (I); and optionally purifying Rivaroxaban of formula (I)

Solvent used is an organic solvent selected from the group consisting of aliphatic hydrocarbons such as but not limited to hexane, cyclohexane, heptane and the like, aromatic hydrocarbons such as but not limited to toluene, xylene and the like; amides such as dialkylformamides such as but not limited to dimethyl formamide and dialkylacetamides such as but not limited to dimethyl acetamide and the like; ethers such as but not limited to di-isopropyl ether, methyl tert. Butyl ether and the like, cyclic ethers such as but not limited to tetrahydrofuran and the like, substituted cyclic ethers such as but not limited to 2-methyl tetrahydrofuran and the like, ketones such as but not limited to acetone, methyl ethyl ketone, Methyl iso-butyl ketone (MIBK) and the like dialkylsulfoxides such as but not limited to dimethyl sulfoxide (DMSO) and the like, nitriles such as but limited to acetonitrile, propionitrile and the like, ionic liquids, halogenated aliphatic hydrocarbons such as but not limited to dichloromethane, dichloroethane, chloroform, 1,2-dichloroethane and the like; esters such as but not limited to ethyl acetates, isopropyl acetate, carboxylic acids such as but not limited to acetic acid, formic acid, propionic acid, butanoic acid and water or mixtures thereof.

Preferably, the said solvent may be sulfoxides, amides. More preferably, the said solvent is dimethyl sulfoxide or dimethylformamide.

The base used is selected from organic or inorganic base. The organic base is selected from diisopropylamine, 1,8-Diazabicyclo[5.4.0]undec-7-ene, 1,5-Diazabicyclo[4.3.0]non-5-ene, 4-Dimethylaminopyridine, di-isopropylethylamine, triethylamine, and the like. The inorganic base is selected from the group comprising of alkali metal carbonates, alkali metal bicarbonates or alkali metal hydroxides. Preferably, the base used is triethylamine.

According to another embodiment, the present invention provides a process for preparation of compound of formula (XIII)

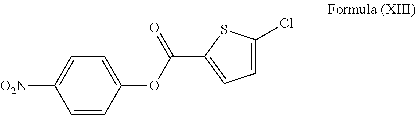 [SMILES: O=C(Oc1ccc([N+](=O)[O-])cc1)c1ccc(Cl)s1]

comprising,
a) reacting compound of formula (XVII)

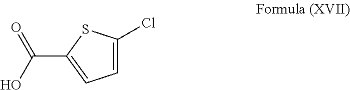

with halogenating agent optionally in a solvent; optionally in presence of a catalyst to obtain compound of formula (XVIII)

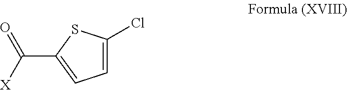

wherein X: halogen;
b) reacting compound of formula (XVIII) with p-nitrophenol, in a solvent, in presence of a base to provide compound of formula (XIII); and optionally purifying compound of formula (XIII).

In a preferred embodiment, purification of compound of formula (XIII) may be carried out by re-crystallization process using solvent at temperature from 0 to reflux temperature of the solvent.

The solvent used in steps (a), (b) and (c) may be same or different; wherein the said solvent is an organic solvent selected from the group comprising aliphatic hydrocarbons such as but not limited to hexane, cyclohexane, heptane and the like, aromatic hydrocarbons such as but not limited to toluene, xylene and the like; amides such as dialkylformamides such as but not limited to dimethyl formamide and dialkylacetamides such as but not limited to dimethyl acetamide and the like; ethers such as but not limited to di-isopropyl ether, methyl tert. Butyl ether and the like, cyclic ethers such as but not limited to tetrahydrofuran and the like, substituted cyclic ethers such as but not limited to 2-methyl tetrahydrofuran and the like, ketones such as but not limited to acetone, methyl ethyl ketone, Methyl iso-butyl ketone (MIBK) and the like dialkylsulfoxides such as but not limited to dimethyl sulfoxide (DMSO) and the like, nitriles such as but limited to acetonitrile, propionitrile and the like, ionic liquids, halogenated aliphatic hydrocarbons such as but not limited to di-chloromethane, dichloroethane, chloroform, 1,2-dichloroethane and the like; esters such as but not limited to acetates, and or mixtures thereof.

Preferably, the solvent used in step (a) may be hydrocarbons, more preferably toluene.

Preferably, the solvent used in step (b) may be hydrocarbons, more preferably methylene dichloride.

Preferably, the solvent used in step (c) may be hydrocarbons or mixture of hydrocarbons, more preferably toluene and n-heptane mixture.

The base used in step (b) is selected from organic or inorganic base. The organic base is selected from diisopropylamine, 1,8-Diazabicyclo[5.4.0]undec-7-ene, 1,5-Diazabicyclo[4.3.0]non-5-ene, 4-Dimethylaminopyridine, di-isopropylethylamine, triethylamine, and the like.

The inorganic, base is selected from the group comprising of alkali metal carbonates, alkali metal bicarbonates or alkali metal hydroxides. Preferably, the base used in step (b) is potassium carbonate

Catalyst used in step (b) may be organic or inorganic catalyst. The organic catalyst is selected from 1,8-Diazabicycloundec-7-ene (DBU) or 1,5-Diazabicyclo(4.3.0)non-5-ene (DBN), dibenzo-18-crwon-6-ether or dimethylaminopyridine, dialkylformamides such as dimethyl formamide and like. The inorganic catalyst is selected from groups comprising alkali metal iodide, iodine, potassium iodide, p-toluene sulfonic acid, sodium iodide, lithium iodide, and the like.

According to yet another embodiment, the present invention provides a process for preparation of Rivaroxaban of formula (I);

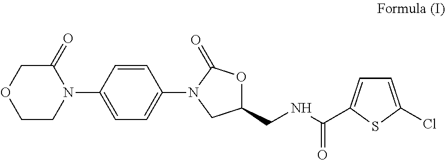 [SMILES: O=C(NC[C@H]1CN(c2ccc(N3CCOCC3=O)cc2)C(=O)O1)c1ccc(Cl)s1]

wherein the said process comprising:
a) reacting compound of formula (VI) or its acid addition salt with compound of formula (XII) in a solvent, optionally in presence of base, optionally in presence of a catalyst to obtain compound of formula (XV); and optionally isolate the compound of formula (XV); and

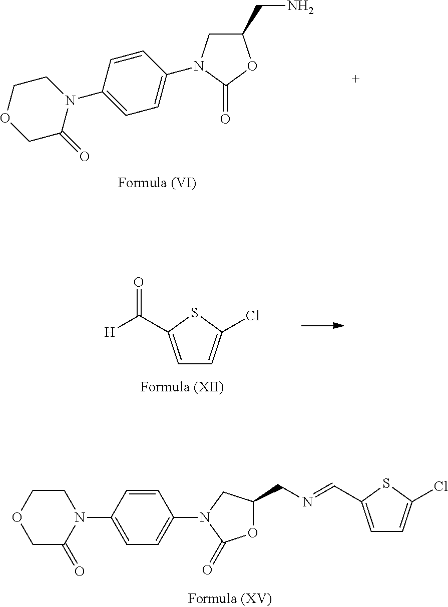 [SMILES: NC[C@@H]1CN(c2ccc(N3CCOCC3=O)cc2)C(=O)O1 ; O=Cc1ccc(Cl)s1 ; O=C1COCCN1c1ccc(N2C[C@@H](C/N=C/c3ccc(Cl)s3)OC2=O)cc1]

b) oxidizing the compound of formula (XV) obtained in step (a) using oxidizing agents, in a solvent, optionally in presence of base, optionally in presence of a catalyst to obtain Rivaroxaban of formula (I); and

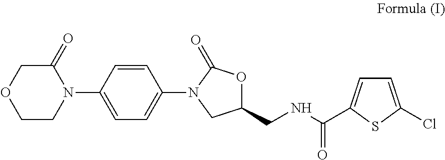 [SMILES: O=C(NC[C@H]1CN(c2ccc(N3CCOCC3=O)cc2)C(=O)O1)c1ccc(Cl)s1]

c) optionally purifying Rivaroxaban compound of formula (I).

The solvent used in step (a), (b) and (c) may be either same or different; wherein the solvent used is an organic solvent selected from the group comprising aliphatic hydrocarbons such as but not limited to hexane, cyclohexane, heptane and the like, aromatic hydrocarbons such as but not limited to toluene, xylene and the like; amides such as dialkylformamides such as but not limited to dimethyl formamide and dialkylacetamides such as but not limited to dimethyl acetamide and the like; ethers such as but not limited to di-isopropyl ether, methyl tert. Butyl ether and the like, cyclic ethers such as but not limited to tetrahydrofuran and the like, substituted cyclic ethers such as but not limited to 2-methyl tetrahydrofuran and the like, ketones such as but not limited to acetone, methyl ethyl ketone, Methyl iso-butyl ketone (MIBK) and the like dialkylsulfoxides such as but not limited to dimethyl sulfoxide (DMSO) and the like, nitriles such as but limited to acetonitrile, propionitrile and the like, ionic liquids, halogenated aliphatic hydrocarbons such as but not limited to di-chloromethane, dichloroethane, chloroform, 1,2-dichloroethane and the like; esters such as but not limited to acetates, alcohols such as but not limited to methanol, ethanol, isopropanol, butanol and the like, carboxylic acids such as but not limited to acetic acid, formic acid, propionic acid, butanoic acid and the like and water or mixtures thereof. Preferably, the said solvent used in step (a) may be dialkylsulfoxide, nitriles, alcohols or mixture thereof. More preferably, the said solvent is dimethylsulfoxide, acetonitrile, methanol or mixtures thereof.

The base used in step (a) and step (b) may be either same or different; wherein the said base is selected from organic or inorganic base.

The organic base is selected from diisopropylamine, di-isopropylethylamine, triethylamine, and the like

The inorganic base is selected from the group comprising of alkali metal carbonates, alkali metal bicarbonates or alkali metal hydroxides.

Catalyst used in step (a) and (b) may be either same or different, wherein the catalyst may be organic, inorganic or phase transfer catalyst. The organic catalyst is selected from 1,8-Diazabicycloundec-7-ene (DBU) or 1,5-Diazabicyclo(4.3.0)non-5-ene (DBN), dibenzo-18-crwon-6-ether or dimethylaminopyridine and like. The inorganic catalyst is selected from groups comprising alkali metal iodide, iodine, potassium iodide, p-toluene sulfonic acid, sodium iodide, lithium iodide, and the like. Phase transfer catalyst is selected from tertiary alkyl ammonium halide and the like.

The oxidizing agent used in step (b) is selected from hydrogen peroxide, peracids including but not limited to peracetic acid, perbenzoic acid, metachloroperbenzoic acid, alkyl hydroperoxides such as but not limited to tertiary butyl hydrogen peroxide and the like, silver iodide, copper iodide, and mixture thereof and the like.

According to yet another embodiment, the present invention provides a process for preparation of Rivaroxaban of formula (I);

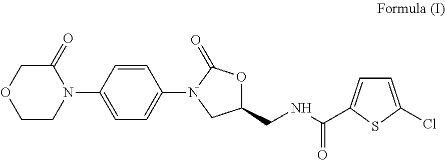 [SMILES: O=C(NC[C@H]1CN(c2ccc(N3CCOCC3=O)cc2)C(=O)O1)c1ccc(Cl)s1]

Wherein the said process comprising:
a) oxidizing compound of formula (XVII) using an oxidizing agent to obtain compound of formula (XII) in a solvent and optionally in the presence of a catalyst;

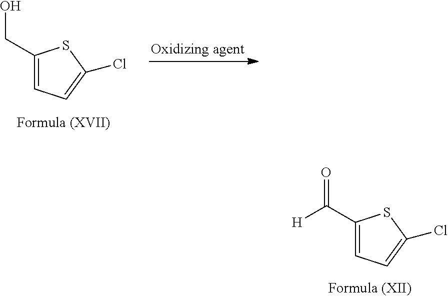 [SMILES: OCc1ccc(Cl)s1 ; O=Cc1ccc(Cl)s1]

b) reacting compound of formula (XII) in situ obtained in step (a) with compound of formula (VI) or its acid addition salt in a solvent, optionally in the presence of a base and optionally in presence of a catalyst to obtain compound of formula (XV) and optionally isolate the compound of formula (XV); and

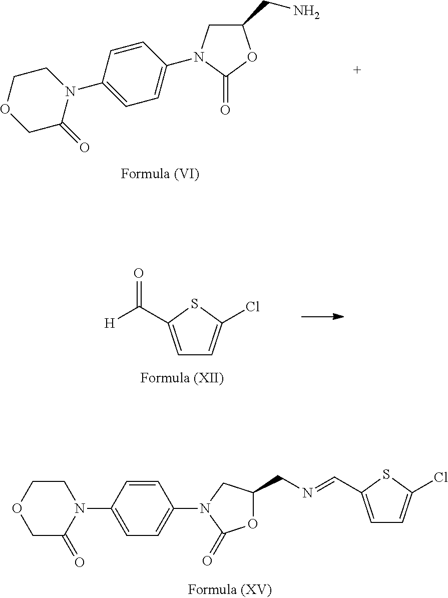 [SMILES: NC[C@@H]1CN(c2ccc(N3CCOCC3=O)cc2)C(=O)O1 ; O=Cc1ccc(Cl)s1 ; O=C1COCCN1c1ccc(N2C[C@@H](C/N=C/c3ccc(Cl)s3)OC2=O)cc1]

c) oxidizing the compound of formula (XV) obtained in step (b) using an oxidizing agents, in a solvent and optionally in presence of a base and optionally in presence of a catalyst to obtain Rivaroxaban of formula (I); and

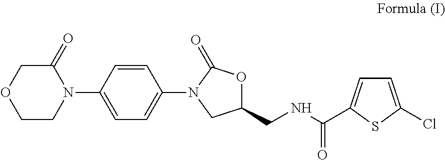 [SMILES: O=C(NC[C@H]1CN(c2ccc(N3CCOCC3=O)cc2)C(=O)O1)c1ccc(Cl)s1]

d) optionally purifying compound of formula (I).

The solvent used in step (a), (b), (c) and (d) may be either same or different; wherein the solvent used is an organic solvent selected from the group comprising aliphatic hydrocarbons such as but not limited to hexane, cyclohexane, heptane and the like, aromatic hydrocarbons such as but not limited to toluene, xylene and the like; amides such as dialkylformamides such as but not limited to dimethyl formamide and dialkylacetamides such as but not limited to dimethyl acetamide and the like; ethers such as but not limited to di-isopropyl ether, methyl tert. Butyl ether and the like, cyclic ethers such as but not limited to tetrahydrofuran and the like, substituted cyclic ethers such as but not limited to 2-methyl tetrahydrofuran and the like, ketones such as but not limited to acetone, methyl ethyl ketone, Methyl iso-butyl ketone (MIBK) and the like dialkylsulfoxides such as but not limited to dimethyl sulfoxide (DMSO) and the like, nitriles such as but limited to acetonitrile, propionitrile and the like, ionic liquids, halogenated aliphatic hydrocarbons such as but not limited to di-chloromethane, dichloroethane, chloroform, 1,2-dichloroethane and the like; esters such as but not limited to acetates, alcohols such as but not limited to methanol, ethanol, isopropanol, butanol and the like, carboxylic acids such as but not limited to acetic acid, formic acid, propionic acid, butanoic acid and the like and water or mixtures thereof.

Preferably, the said solvent used in step (a) may be nitriles, hydrocarbons, water or mixture thereof. More preferably, the said solvent is acetonitrile, dichloromethane, dichloroethane, water or mixtures thereof.

Preferably, the said solvent used in step (b) and (c) may be nitriles, water or mixture thereof. More preferably, the said solvent is acetonitrile, water or mixtures thereof.

Preferably, the said solvent used in step (d) may be dialkylsulfoxide, nitriles, alcohols, water or mixture thereof. More preferably, the said solvent is dimethyl sulfoxide, acetonitrile, alcohol or mixtures thereof.

The base used in step (b) and step (c) may be either same or different; wherein the said base is selected from organic or inorganic base.

The organic base is selected from diisopropylamine, 1,8-Diazabicyclo[5.4.0]undec-7-ene, 1,5-Diazabicyclo[4.3.0]non-5-ene, 4-Dimethylaminopyridine, di-isopropylethylamine, triethylamine, and the like

The inorganic base is selected from the group comprising of alkali metal carbonates, alkali metal bicarbonates or alkali metal hydroxides.

Catalyst used in steps (a), (b) and (c) may be either same or different, wherein the catalyst may be organic, inorganic or phase transfer catalyst.

Catalyst used in step (b) may be organic or inorganic catalyst.

The organic catalyst is selected from 1,8-Diazabicycloundec-7-ene (DBU) or 1,5-Diazabicyclo(4.3.0)non-5-ene (DBN), dibenzo-18-crwon-6-ether or dimethylaminopyridine, dialkylformamides such as dimethyl formamide and like.

The inorganic catalyst is selected from groups comprising alkali metal iodide, iodine, potassium iodide, p-toluene sulfonic acid, sodium iodide, lithium iodide, and the like.

The oxidizing agent used in steps (a) and (c) may be same or different; wherein the said oxidizing agent is selected from hydrogen peroxide, peracids including but not limited to peracetic acid, perbenzoic acid, metachloroperbenzoic acid, alkyl hydroperoxides such as but not limited to tertiary butyl hydrogen peroxide and the like, silver iodide, copper iodide, and mixture thereof and the like.

According to another embodiment, the present invention provides a compound of formula (XIII)

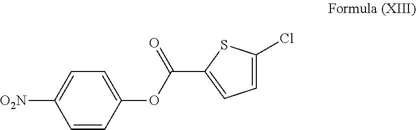 [SMILES: O=C(Oc1ccc([N+](=O)[O-])cc1)c1ccc(Cl)s1]

According to another embodiment, the present invention provides a compound of formula (XV)

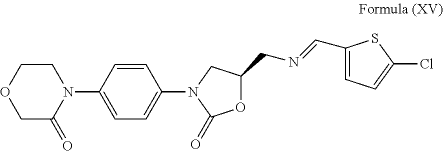 [SMILES: O=C1COCCN1c1ccc(N2C[C@@H](C/N=C/c3ccc(Cl)s3)OC2=O)cc1]

According to another embodiment, the present invention provides a compound of formula (IX),

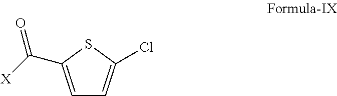

Wherein; X may be sulfonyloxy, imidazole, triazole, tetrazole, alkoxy, substituted alkoxy, tri-halomethoxy, N-hydroxysuccinamide, p-nitrophenol, N-hydroxythalamide, N-hydroxybenzotriazole, or OR;

Wherein; R may be alkyl, aryl and substituted aryl.

Rivaroxaban prepared according to any of the processes of the present invention has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

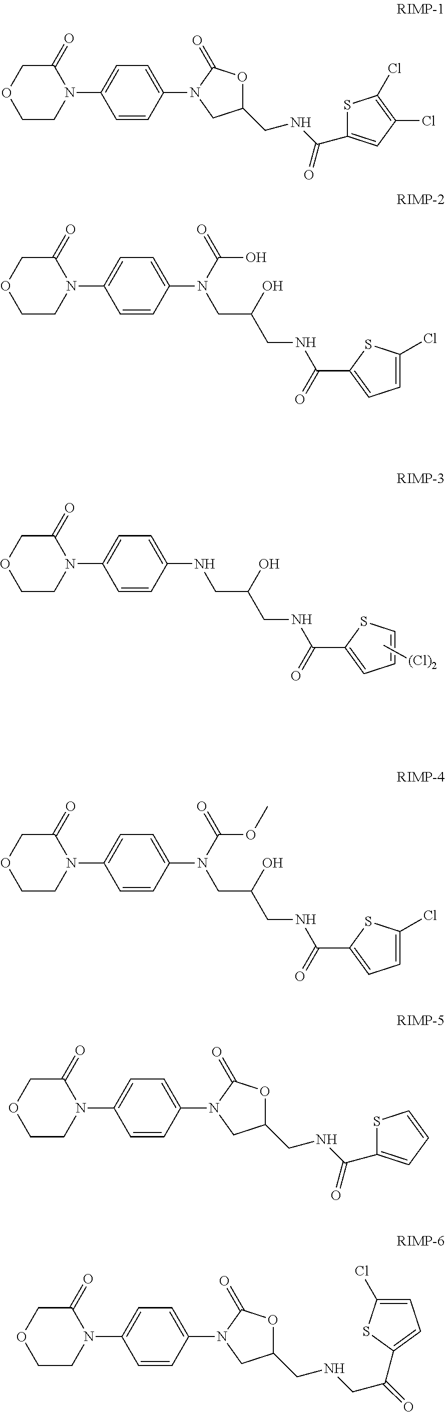

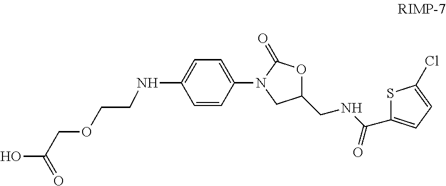 [SMILES: O=C(O)COCCNc1ccc(N2CC(CNC(=O)c3ccc(Cl)s3)OC2=O)cc1]

According to any of the preceding embodiments of the present invention, Rivaroxaban of compound of formula (I) or its pharmaceutically acceptable salts may be purified as per the process known in the art; wherein Rivaroxaban may be purified either by re-crystallization, re-saltification, washing with solvent.

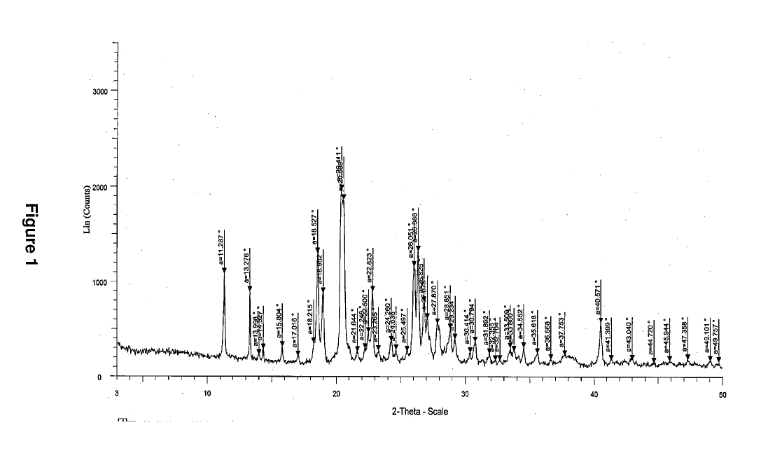

FIG. 1 illustrates X-ray powder diffraction (XRD) pattern of hydrochloride salt of compound of formula (VI), prepared according to example 3. It demonstrates the crystalline nature of hydrochloride salt of compound of formula (VI). The X-ray diffractogram was measured on Bruker Axe, DS advance Power X-ray Diffractometer with Cu K alpha-1 Radiation source having the wavelength 1.541 Å.

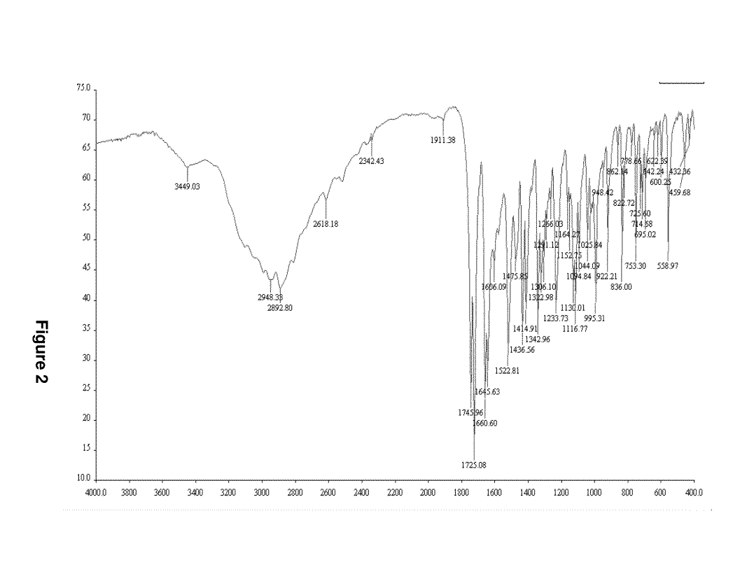

FIG. 2 illustrates Infrared spectrum (IR) of hydrochloride salt of compound of formula (VI), prepared according to example 3. The IR spectrum of crystalline form of hydrochloride salt of compound of formula (VI) having characteristic peaks at 432.36, 459.68, 558.97, 600.25, 753.30, 836.0, 922.21, 995.31, 1044.09, 1116.77, 1130.01, 1233.73, 1342.96, 1414.91, 1436.56, 1475.85, 1522.81, 1645.63, 1660.60, 1725.08, 1745.96, 1911.38, 2618.18, 2892.80, 2948.33 cm⁻¹. The IR spectra of hydrochloride salt of compound of formula (VI) of the invention has been recorded on a Fourier Transform Infrared Spectroscopy, Perkin Elmer model 100 instrument using potassium bromide pellet method.

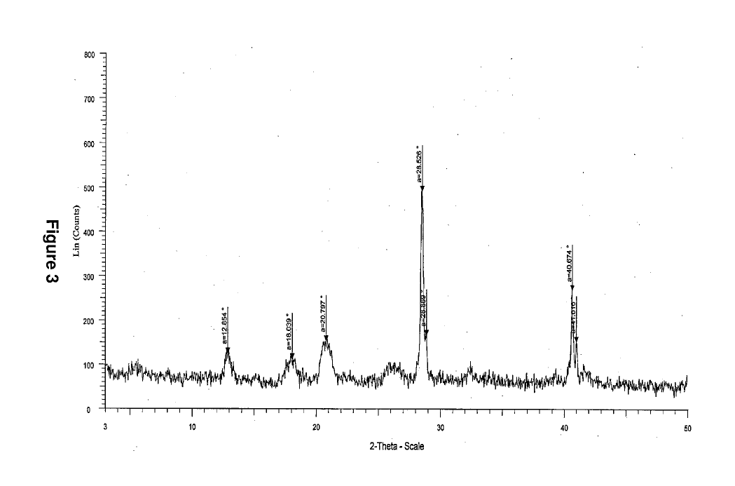

FIG. 3 illustrates X-ray powder diffraction (XRD) pattern of compound of formula (VI) as a free base, prepared according to example 6. It demonstrates the crystalline nature of compound of formula (VI) as a free base. The X-ray diffractogram was measured on Bruker Axe, DS advance Power X-ray Diffractometer with Cu K alpha-1 Radiation source having the wavelength 1.541 Å.

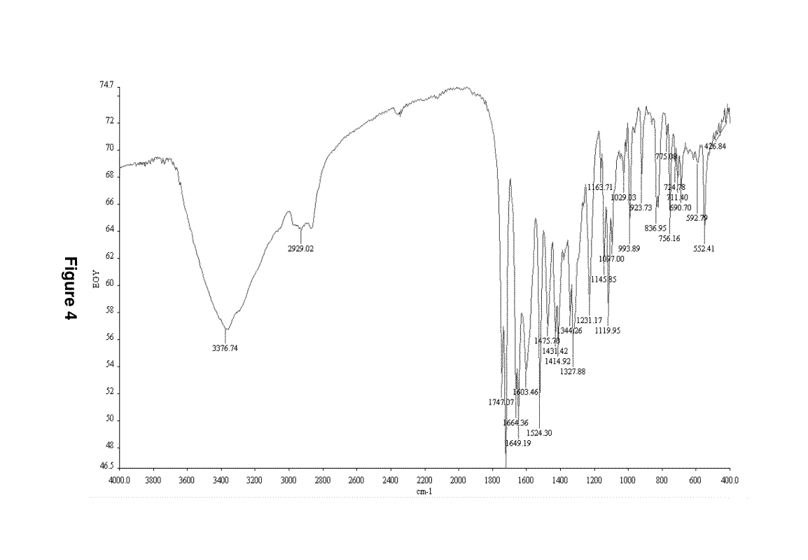

FIG. 4 illustrates Infrared spectrum (IR) of compound of formula (VI) as a free base, prepared according to example 6. The IR spectrum of crystalline form of compound of formula (VI) as a free base having characteristic peaks at 552.41, 756.16, 836.95, 923.73, 993.89, 1119.95, 1145.85, 1231.17, 1327.88, 1344.26, 1524.30, 1603.46, 1649.19, 1664.36, 1723.87, 1747.07, 3376.74 cm⁻¹. The IR spectra of compound of formula (VI) as a free base of the invention has been recorded on a Fourier Transform Infrared Spectroscopy, Perkin Elmer model 100 instrument using potassium bromide pellet method.

### BEST MODE OR EXAMPLES FOR WORKING OF THE INVENTION

The present invention is described in the examples given below; further these are provided only to illustrate the invention and therefore should not be construed to limit the scope of the invention.

### Example-1

### Preparation of 2-[(2R)-2-hydroxy-3-{[4-(3-oxomorpholin-4-yl)phenyl]amino}propyl]-1H-isoindole-1,3(2H)-dione

A suspension of 4-(4-aminophenyl)morpholin-3-one (100 gm) and 2-[(2S)-oxiran-2-ylmethyl]-1H-isoindole-1,3(2H)-dione (116.2 gm) in isopropyl alcohol and water mixture (1700 ml:300 ml) is refluxed for 25-30 h. The precipitated solid is filtered off, washed with isopropyl alcohol (100 ml) to obtain solid, which is then dried under vacuum at 50 to 55° C. for 4 to 5 hr to obtain 2-[(2R)-2-hydroxy-3-{[4-(3-oxomorpholin-4-yl)phenyl]amino}propyl]-1H-isoindole-1,3(2H)-dione. [Yield=170 gm (82.6%); Purity (HPLC)=95.0%]

### Example-2

### Preparation of 2-({(5S)-2-oxo-3-[4-(3-oxomorpholin-4-yl)phenyl]-1,3-oxazolidin-5-yl}methyl)-1H-isoindole-1,3(2H)-dione

To suspension of 2-[(2R)-2-hydroxy-3-{[4-(3-oxomorpholin-4-yl)phenyl]amino}propyl]-1H-isoindole-1,3(2H)-dione (170 gm) and potassium carbonate (59.3 gm) in dichloromethane (1500 ml) was added 1,1′-carbonylbis(1H-imidazole) (153.4 gm) at room temperature. Reaction mass was then stirred for 5 hr at room temperature. After completion of reaction, inorganic base is removed by filtration. The obtained filtrate is concentrated under reduced pressure to yield solid. To this solid tetrahydrofuran (850 ml) was added followed by stirring and filtration. The obtained solid is dried under vacuum for 4 hr at 50° C. to obtain 2-({(5S)-2-oxo-3-[4-(3-oxomorpholin-4-yl)phenyl]-1,3-oxazolidin-5-yl}methyl)-1H-isoindole-1,3(2H)-dione. [Yield=160 gm (88.2%); Purity (HPLC)=99.65%]

### Example-3

### Preparation of 4-{4-[(5S)-5-(aminomethyl)-2-oxo-1,3-oxazolidin-3-yl]phenyl}morpholin-3-one hydrochloride

Methylamine (40% strength, 92 ml) is added lot-wise to suspension of 2-({(5S)-2-oxo-3-[4-(3-oxomorpholin-4-yl)phenyl]-1,3-oxazolidin-5-yl}methyl)-1H-isoindole-1,3(2H)-dione (150 gm) in mixture of methanol (750 ml) & water (375 ml) at 25 to 30° C. over a period of 2 hr. The reaction mass was then refluxed for 6 hr. Reaction mass was concentrated under reduced pressure to obtain syrup. Mixture of dichloromethane (750 ml) and methanol (150 ml) was charged to syrup. pH of the reaction mass was adjusted to 2-3 using isopropyl alcohol-hydrochloric acid. Reaction mass was stirred for 1 hr & filtered off the solid. The solid obtained was washed with dichloromethane (50 ml) and dried under vacuum at 50 to 55° C. for 5 hr to obtain 4-{4-[(5S)-5-(aminomethyl)-2-oxo-1,3-oxazolidin-3-yl]phenyl}morpholin-3-one hydrochloride.

Crystalline form of hydrochloride salt of compound of formula (VI) is characterized by its XPRD pattern as per FIG. 1

The IR spectrum of crystalline form of hydrochloride salt of compound of formula (VI) having characteristic peaks at 432.36, 459.68, 558.97, 600.25, 753.30, 836.0, 922.21, 995.31, 1044.09, 1116.77, 1130.01, 1233.73, 1342.96, 1414.91, 1436.56, 1475.85, 1522.81, 1645.63, 1660.60, 1725.08, 1745.96, 1911.38, 2618.18, 2892.80, 2948.33 cm⁻¹ (FIG. 2) [Yield=70 gm (60%); Purity (HPLC)=95.85%]

### Example-4

### Preparation of 4-{4-[(5S)-5-(aminomethyl)-2-oxo-1,3-oxazolidin-3-yl]phenyl}morpholin-3-one hydrochloride

Methylamine (40% strength, 153.2 ml) is added to suspension of 2-({(5S)-2-oxo-3-[4-(3-oxomorpholin-4-yl)phenyl]-1,3-oxazolidin-5-yl}methyl)-1H-isoindole-1,3(2H)-dione (150 gm) in methanol (1500 ml) at 25 to 30° C. The reaction mass was then refluxed for 6 hr. After completion of reaction, the reaction mass was cooled to 25-30° C., the pH of reaction mass was adjusted to 2-3 using conc. HCl. Reaction mass was stirred for 1 hr & filtered off the solid. The solid obtained was washed with methanol (50 ml)

Obtained solid was dissolved in mixture of methanol (500 ml) and methylene dichloride (750 ml) by adjusting pH of reaction mass to 7-8 using triethylamine at 25-30° C. and stirred till clear solution was obtained, pH of reaction mass was adjusted to 4-5 by using acetic acid at 25-30° C. Reaction mass was stirred for 1 h at 25-30° C. and filtered off the solid. The solid obtained was washed with methanol (50 ml) and dried under vacuum at 50 to 55° C. for 5 hr to obtain 4-{4-[(5S)-5-(aminomethyl)-2-oxo-1,3-oxazolidin-3-yl]phenyl}morpholin-3-one hydrochloride.

[Yield=92.4 gm (80%); Purity (HPLC)=98.0%]

### Example-5

### Preparation of 4-{4-[(5S)-5-(aminomethyl)-2-oxo-1,3-oxazolidin-3-yl]phenyl}morpholin-3-one hydrochloride

Methylamine (40% strength, 153.2 ml) was added to suspension of 2-({(5S)-2-oxo-3-[4-(3-oxomorpholin-4-yl)phenyl]-1,3-oxazolidin-5-yl}methyl)-1H-isoindole-1,3(2H)-dione (150 gm) in methanol (1500 ml) at 25 to 30° C. The reaction mass was refluxed for 6 hr, cooled to 25-30° C., followed by slow addition of acetic acid (300 ml) reaction mass. Thereaction mass was stirred for 30 min at 25-30° C. pH of the reaction mass was adjusted to 2-3 using conc. HCl. Reaction mass was stirred for 1 hr & filtered off the solid. The solid obtained was washed with methanol (50 ml)

Obtained solid dissolved in mixture of methanol (500 ml) and methylene dichloride (750 ml) by adjusting pH of reaction mass to 7-8 using triethylamine at 25-30° C. and stirred till clear solution was obtained, then adjusted the pH of reaction mass to 4-5 by using acetic acid at 25-30° C. Reaction mass was stirred for 1 h at 25-30° C. and filtered off the solid. The solid obtained was washed with methanol (50 ml) and dried under vacuum at 50 to 55° C. for 5 hr to obtain 4-{4-[(5S)-5-(aminomethyl)-2-oxo-1,3-oxazolidin-3-yl]phenyl}morpholin-3-one hydrochloride.

[Yield=92.4 gm (80%); Purity (HPLC)=98.0%]

### Example-6

### Preparation of 4-{4-[(5S)-5-(aminomethyl)-2-oxo-1,3-oxazolidin-3-yl]phenyl}morpholin-3-one base

4-{4-[(5S)-5-(aminomethyl)-2-oxo-1,3-oxazolidin-3-yl]phenyl}morpholin-3-one hydrochloride (5 g) was stirred in the mixture of methanol (25 ml) and potassium carbonate (3.16) for 30 minutes at 25 to 30° C. The inorganic salt was filtered. The obtained filtrate was concentrated under reduced pressure to obtain solid 4-{4-[(5S)-5-(aminomethyl)-2-oxo-1,3-oxazolidin-3-yl]phenyl}morpholin-3-one base.

Crystalline form of compound of formula (VI) as a free base is characterized by its XPRD pattern as per FIG. 3.

The IR spectrum of crystalline form of compound of formula (VI) as a free base having characteristic peaks at 552.41, 756.16, 836.95, 923.73, 993.89, 1119.95, 1145.85, 1231.17, 1327.88, 1344.26, 1524.30, 1603.46, 1649.19, 1664.36, 1723.87, 1747.07, 3376.74 cm⁻¹ (FIG. 4). [Yield=2.5 gm (56%)]

### Example-7

### Preparation of 5-chloro-N-({(5S)-2-oxo-3-[4-(3-oxomorpholin-4-yl)phenyl]-1,3-oxazolidin-5-yl}methyl)thiophene-2-carboxamide (Rivaroxaban)

At room temperature, 5-chlorothiohene-2-carbonyl chloride (26.5 gm)) was added drop-wise to a solution of 4-{4-[(5S)-5-(aminomethyl)-2-oxo-1,3-oxazolidin-3-yl]phenyl}morpholin-3-one hydrochloride (41 gm) & potassium carbonate (42 gm) in dichloromethane (200 ml). The reaction mass was stirred at 25 to 30° C. for 5 hr. After completion of reaction, water (200 ml) was added to reaction mass followed by addition of hydrochloric acid (10 ml). The precipitated solid was then filtered and washed with water (100 ml) to obtain solid which was then dried under vacuum at 55 to 60° C. for 5 to 6 hr to obtain 5-chloro-N-({(5S)-2-oxo-3-[4-(3-oxomorpholin-4-yl)phenyl]-1,3-oxazolidin-5-yl}methyl)thiophene-2-carboxamide

[Yield=48 gm (88%); Purity (HPLC)=99.0%]

### Example-8

### Preparation of 5-chloro-N-({(5S)-2-oxo-3-[4-(3-oxomorpholin-4-yl)phenyl]-1,3-oxazolidin-5-yl}methyl)thiophene-2-carboxamide (Rivaroxaban)

4-nitrophenyl 5-chlorothiophene-2-carboxylate (2.33 gm) was added to a solution of 4-{4-[(5S)-5-(aminomethyl)-2-oxo-1,3-oxazolidin-3-yl]phenyl}morpholin-3-one (2 gm) and potassium carbonate (1.13 gm) in dichloromethane (20 ml) at 25 to 30° C. After completion of reaction, water and hydrochloric acid was added to the reaction mass & concentrated reaction mass under reduced pressure to obtain crude solid. Methanol was charged to the obtained crude solid and stirred for 30 minutes at 60° C. Finally filtered the solid and dried at 50 to 55° C. for 3 to 5 hr to obtain 5-chloro-N-({(5S)-2-oxo-3-[4-(3-oxomorpholin-4-yl)phenyl]-1,3-oxazolidin-5-yl}methyl)thiophene-2-carboxamide

[Yield=1.5 gm (55.5%); Purity (HPLC)=97.98%]

### Example-9

### Preparation of 5-chloro-N-({(5S)-2-oxo-3-[4-(3-oxomorpholin-4-yl)phenyl]-1,3-oxazolidin-5-yl}methyl)thiophene-2-carboxamide (Rivaroxaban)

4-nitrophenyl 5-chlorothiophene-2-carboxylate (9.5 gm) was added to a solution of 4-{4-[(5S)-5-(aminomethyl)-2-oxo-1,3-oxazolidin-3-yl]phenyl}morpholin-3-one hydrochloride (10 gm) in dimethyl sulfoxide (40 ml) and triethyl amine (4.6 gm) at 35 to 40° C.

After completion of reaction, acetonitrile (40 ml) was added to the reaction mass. Heat the reaction mass to reflux or till clear solution appear, added methanol (60 ml), cool the reaction mass to 25 to 30° C., stir the reaction mass for 2 h, filtered the solid and dried at 50 to 55° C. for 3 to 5 hr to obtain 5-chloro-N-({(5S)-2-oxo-3-[4-(3-oxomorpholin-4-yl)phenyl]-1,3-oxazolidin-5-yl}methyl)thiophene-2-carboxamide

[Yield=11.0 gm (82.7%); Purity (HPLC)=99.7%]

We claim:

1. An improved process for preparation of Rivaroxaban of formula (I), the process comprising:
a) reacting, 4-(4-aminophenyl)morpholine-3-one of formula (II) with 2-[(2S)-oxiran-2-ylmethyl]-1H-isoindole-1,3(2H)-dione of formula (III) in a first solvent to obtain 2-[(2R)-2-hydroxy-3-{[4-(3-oxomorpholin-4-yl)phenyl]amino}propyl]-1H-isoindole-1,3(2H)-dione of formula (IV); 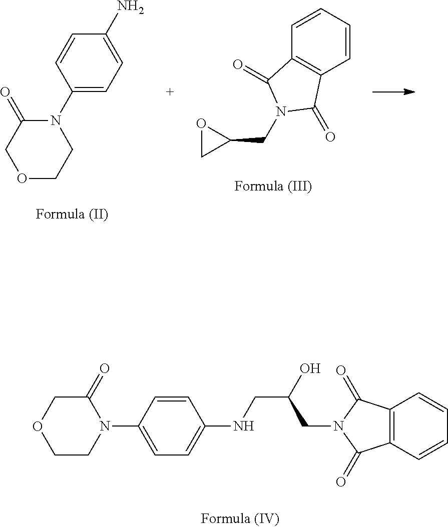 [SMILES: Nc1ccc(N2CCOCC2=O)cc1 ; O=C1c2ccccc2C(=O)N1C[C@H]1CO1 ; O=C1c2ccccc2C(=O)N1C[C@H](O)CNc1ccc(N2CCOCC2=O)cc1]
b) preparing compound of formula (V) by reacting compound of formula (IV) using a phosgene or phosgene equivalent(s) or anhydrides or bis(aryl) carbonate of formula (XVI) 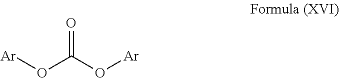
wherein, Ar can be phenyl, substituted phenyl, selected from p-nitrophenyl, 4-(trifluoromethyl)phenyl, 4-cyanophenyl and the like,
in a second solvent and optionally in presence of a base; 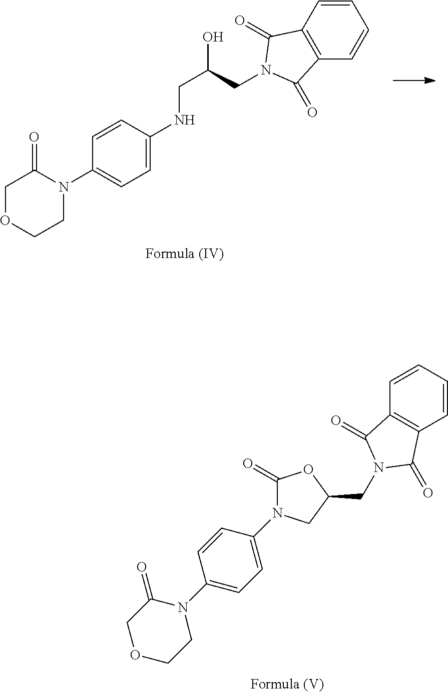 [SMILES: O=C1c2ccccc2C(=O)N1C[C@H](O)CNc1ccc(N2CCOCC2=O)cc1 ; O=C1c2ccccc2C(=O)N1C[C@H]1CN(c2ccc(N3CCOCC3=O)cc2)C(=O)O1]
c) eliminating the pthalamide group from compound of formula (V) in the said first solvent using a de-protecting agent to obtain 4-{4-[(5S)-5-(aminomethyl)-2-oxo-1,3-oxazolidin-3-yl]phenyl}morpholin-3-one formula (VI); optionally isolating and purifying compound of formula (VI); optionally converting to its acid addition salt; 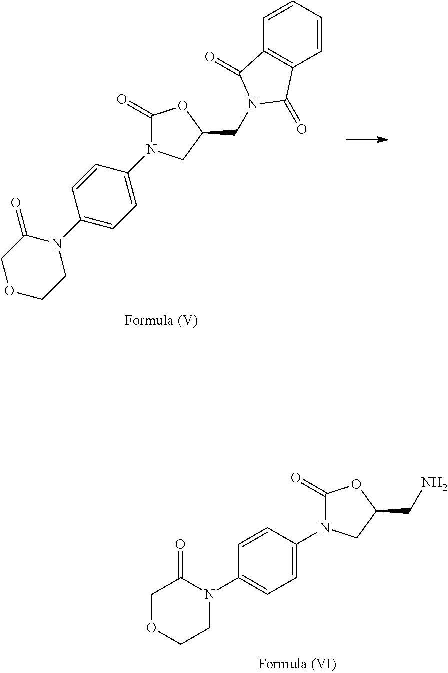 [SMILES: O=C1c2ccccc2C(=O)N1C[C@H]1CN(c2ccc(N3CCOCC3=O)cc2)C(=O)O1 ; NC[C@H]1CN(c2ccc(N3CCOCC3=O)cc2)C(=O)O1]
d) reacting compound of formula (VI) or acid addition salt with 5-chlorothiophene-2-carbonyl chloride of formula (VIII) in a third solvent and in presence of a base to obtain Rivaroxaban of formula (I), and isolating compound of formula (I); or optionally, the reaction can be carried out in biphasic medium and optionally in the presence of a phase transfer catalyst. 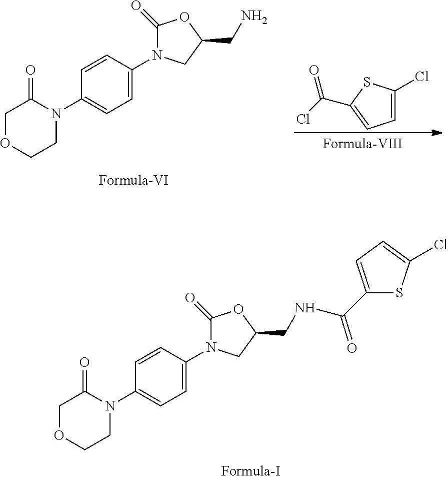 [SMILES: NC[C@H]1CN(c2ccc(N3CCOCC3=O)cc2)C(=O)O1 ; O=C(Cl)c1ccc(Cl)s1 ; O=C(NC[C@H]1CN(c2ccc(N3CCOCC3=O)cc2)C(=O)O1)c1ccc(Cl)s1]
e) optionally, purifying Rivaroxaban of formula (I).

2. The process of claim 1, wherein the first solvent used in step (a) and step (c) may be either same or different; wherein the said solvent is an organic solvent selected from the group consisting of aliphatic hydrocarbons, aromatic hydrocarbons, amides including but not limited to dialkylformamides and dialkylacetamides, ethers, cyclic ethers, substituted cyclic ethers, alcohols, ketones, dialkylsulfoxides, nitriles, ionic liquids, halogenated aliphatic hydrocarbons, water or mixtures thereof.

3. The process of claim 1, wherein the second solvent used in step (b) is an organic solvent selected from the group consisting of aliphatic hydrocarbons, aromatic hydrocarbons, amides including but not limited to dialkylformamides and dialkylacetamides, ethers, cyclic ethers, substituted cyclic ethers, ketones, dialkylsulfoxides, nitriles, ionic liquids, halogenated aliphatic hydrocarbons or mixtures thereof.

4. The process of claim 1, wherein the third solvent used in step (d) is an organic solvent selected from the group consisting of aliphatic hydrocarbons, aromatic hydrocarbons, amides including but not limited to dialkylformamides and dialkylacetamides, ethers, cyclic ethers, substituted cyclic ethers, ketones, dialkylsulfoxides, nitriles, ionic liquids, halogenated aliphatic hydrocarbons, water or mixtures thereof.

5. The process of claim 1, wherein the base used in step (b) and step (d) is selected from organic base including but not limited to diisopropylamine, 1,8-diazabicyclo[5.4.0]undec-7-ene,1,5-diazabicyclo[4.3.0]non-5-ene,4-dimethylaminopyridine, diisopropylethylamine or triethylamine; or inorganic base selected from group consisting of alkali metal carbonates, alkali metal bicarbonates or alkali metal hydroxides.

6. A process for preparation of Rivaroxaban of formula (I), the process comprising: 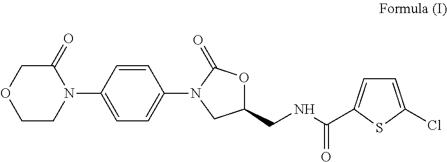 [SMILES: O=C(NC[C@H]1CN(c2ccc(N3CCOCC3=O)cc2)C(=O)O1)c1ccc(Cl)s1]
reacting compound of formula (VI) or its acid addition salt 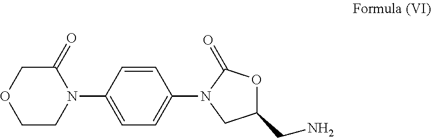 [SMILES: NC[C@H]1CN(c2ccc(N3CCOCC3=O)cc2)C(=O)O1]
with compound of formula (IX) 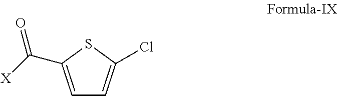
wherein; X may be sulfonyloxy, imidazole, triazole, tetrazole, alkoxy, substituted alkoxy, tri-halomethoxy, N-hydroxysuccinamide, p-nitrophenol, N-hydroxythalamide, N-hydroxybenzotriazole, or OR;
wherein; R may be alkyl, aryl and substituted aryl;
in a solvent and in presence of a base to provide Rivaroxaban of formula (I); and
optionally purifying Rivaroxaban of formula (I).

7. The process of claim 6, wherein the solvent is an organic solvent selected from the group consisting of aliphatic hydrocarbons, aromatic hydrocarbons, amides including but not limited to dialkylformamides and dialkylacetamides, ethers, cyclic ethers, substituted cyclic ethers, ketones, dialkylsulfoxides, nitriles, ionic liquids, halogenated aliphatic hydrocarbons, esters, water or mixtures thereof.

8. The process of claim 6, wherein base used is selected from organic base including but not limited to diisopropylamine, 1,8-diazabicyclo[5.4.0]undec-7-ene, 1,5-diazabicyclo[4.3.0]non-5-ene, 4-dimethylaminopyridine, di-isopropylethylamine or triethylamine; or inorganic base selected from group consisting of alkali metal carbonates, alkali metal bicarbonates or alkali metal hydroxides.

9. A process for preparation of Rivaroxaban of formula (I), the process comprising: 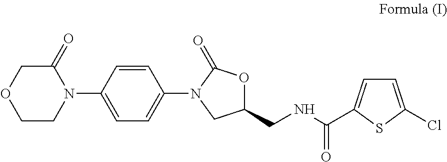 [SMILES: O=C(NC[C@H]1CN(c2ccc(N3CCOCC3=O)cc2)C(=O)O1)c1ccc(Cl)s1]
a) reacting compound of formula (VI) or its acid addition salt with compound of formula (XII) in a solvent, optionally in presence of base, optionally in presence of a catalyst to obtain compound of formula (XV); and optionally isolate the compound of formula (XV); 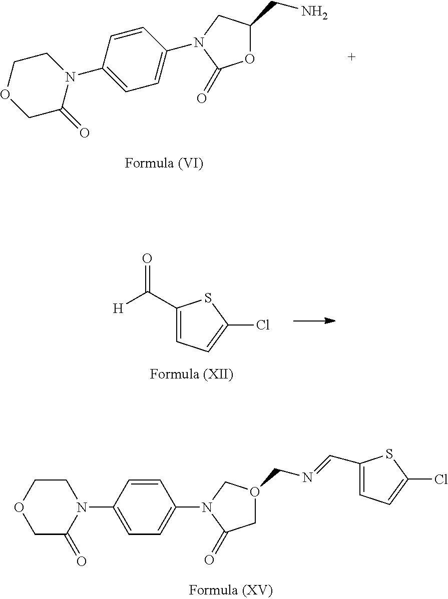
b) oxidizing the compound of formula (XV) obtained in step (a) using an oxidizing agent(s), in a solvent, optionally in presence of a base, optionally in presence of a catalyst to obtain Rivaroxaban of formula (I); and
c) optionally purifying Rivaroxaban compound of formula (I).

10. The process of claim 9, wherein the solvent used in step (a) and (b) may be either same or different; wherein the solvent used is an organic solvent selected from the group consisting of aliphatic hydrocarbons, aromatic hydrocarbons, amides including but not limited to dialkylformamides and dialkylacetamides, ethers, cyclic ethers, substituted cyclic ethers, ketones, dialkylsulfoxides, nitriles, ionic liquids, halogenated aliphatic hydrocarbons, esters, alcohols, water or mixtures thereof.

11. The process of claim 9, wherein base used in step (a) and (b) may be either same or different, and is selected from organic base including but not limited to diisopropylamine, di-isopropylethylamine or triethylamine; or inorganic base selected from group consisting of alkali metal carbonates, alkali metal bicarbonates or alkali metal hydroxides.

12. The process of claim 9, wherein the catalyst used in step (a) and (b) may be either same or different and may be organic catalyst selected from 1,8-diazabicycloundec-7-ene (DBU) or 1,5-diazabicyclo(4.3.0)non-5-ene (DBN), dibenzo-18-crwon-6-ether or 4-dimethylaminopyridine, dialkylformamides such as dimethyl formamide and like; or inorganic catalyst selected from groups consisting of alkali metal iodide, iodine, potassium iodide, p-toluene sulfonic acid, sodium iodide, lithium iodide, and the like; or phase transfer catalyst.

13. The process of claim 9, wherein the oxidizing agent used in step (b) is selected from hydrogen peroxide, peracids, alkyl hydroperoxides including but not limited to tertiary butyl hydrogen peroxide; silver iodide, copper iodide or mixture thereof.

14. A process for preparation of Rivaroxaban of formula (I), the process comprising: 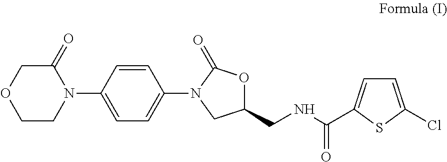 [SMILES: O=C(NC[C@H]1CN(c2ccc(N3CCOCC3=O)cc2)C(=O)O1)c1ccc(Cl)s1]
a) oxidizing compound of formula (XVII) using an oxidizing agent to obtain compound of formula (XII) in a solvent and optionally in presence of a catalyst; 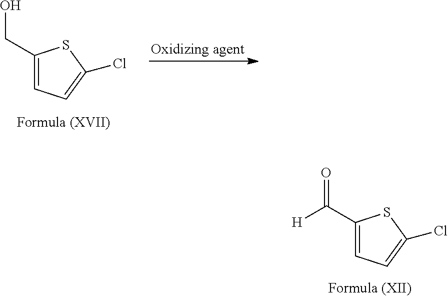 [SMILES: OCc1ccc(Cl)s1 ; O=Cc1ccc(Cl)s1]
b) reacting compound of formula (XII) in situ obtained in step (a) with compound of formula (VI) or its acid addition salt in a solvent, optionally in the presence of a base and optionally in presence of a catalyst to obtain compound of formula (XV) and optionally isolate the compound of formula (XV); and 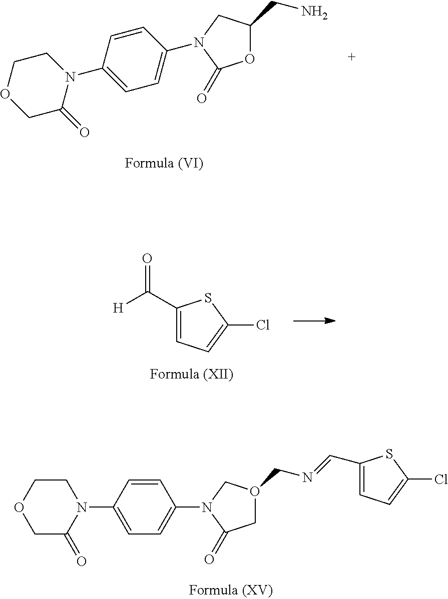
c) oxidizing the compound of formula (XV) obtained in step (b) using an oxidizing agent(s), in a solvent and optionally in presence of a base and optionally in presence of a catalyst to obtain Rivaroxaban of formula (I); and
d) optionally purifying compound of formula (I)

15. The process of claim 14, wherein the said solvent used in steps (a), (b) and (c) may be either same or different; wherein the solvent used is an organic solvent selected from the group consisting of aliphatic hydrocarbons, aromatic hydrocarbons, amides including but not limited to dialkylformamides and dialkylacetamides, ethers, cyclic ethers, substituted cyclic ethers, ketones, dialkylsulfoxides, nitriles, ionic liquids, halogenated aliphatic hydrocarbons, esters, alcohols, water or mixtures thereof.

16. The process of claim 14, wherein base used in steps (b) and (c) may be either same or different, and is selected from organic base including but not limited to diisopropylamine, di-isopropylethylamine or triethylamine; or inorganic base selected from group consisting of alkali metal carbonates, alkali metal bicarbonates or alkali metal hydroxides.

17. The process of claim 14, wherein the catalyst used in step (a), (b) and (c) may be either same or different, and may be organic catalyst selected from 1,8-diazabicycloundec-7-ene (DBU) or 1,5-diazabicyclo(4.3.0)non-5-ene (DBN), dibenzo-18-crwon-6-ether or dimethylaminopyridine, dialkylformamides including but not limited to dimethyl formamide; or inorganic catalyst selected from groups consisting of alkali metal iodide, iodine, potassium iodide, p-toluene sulfonic acid, sodium iodide, lithium iodide, and the like; or phase transfer catalyst.

18. The process of claim 14, wherein the oxidizing agents used in step (a) and (c) may be either same or different, the said oxidizing agent is selected from hydrogen peroxide, peracids including but not limited to peracetic acid, perbenzoic acid or metachloroperbenzoic acid; alkyl hydroperoxides such as but not limited to tertiary butyl hydrogen peroxide, silver iodide, copper iodide, and mixture thereof.

19. Compound of formula (XIII)

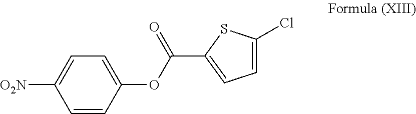 [SMILES: O=C(Oc1ccc([N+](=O)[O-])cc1)c1ccc(Cl)s1]

20. Compound of formula (XV)

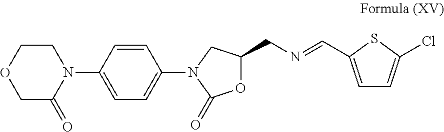 [SMILES: O=C1COCCN1c1ccc(N2C[C@@H](C/N=C/c3ccc(Cl)s3)OC2=O)cc1]

21. Compound of formula (IX) 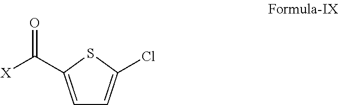
Wherein; X may be sulfonyloxy, imidazole, triazole, tetrazole, alkoxy, substituted alkoxy, tri-halomethoxy, N-hydroxysuccinamide, p-nitrophenol, N-hydroxythalamide, N-hydroxybenzotriazole, or OR;
Wherein; R may be alkyl, aryl and substituted aryl.

22. The process according to claim 1, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

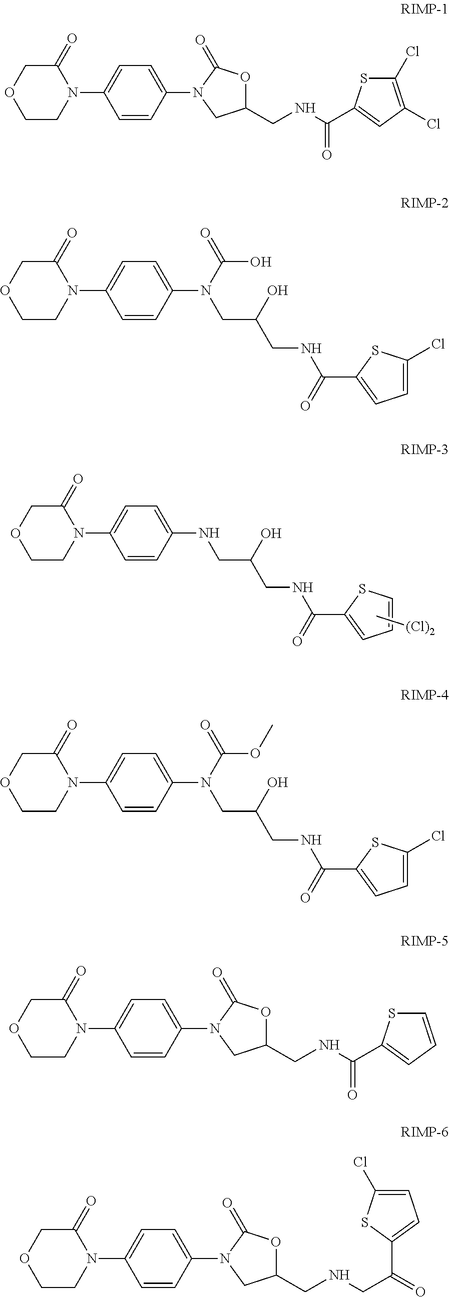

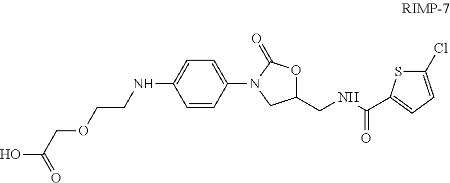 [SMILES: O=C(O)COCCNc1ccc(N2CC(CNC(=O)c3ccc(Cl)s3)OC2=O)cc1]

23. The process according to claim 2, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

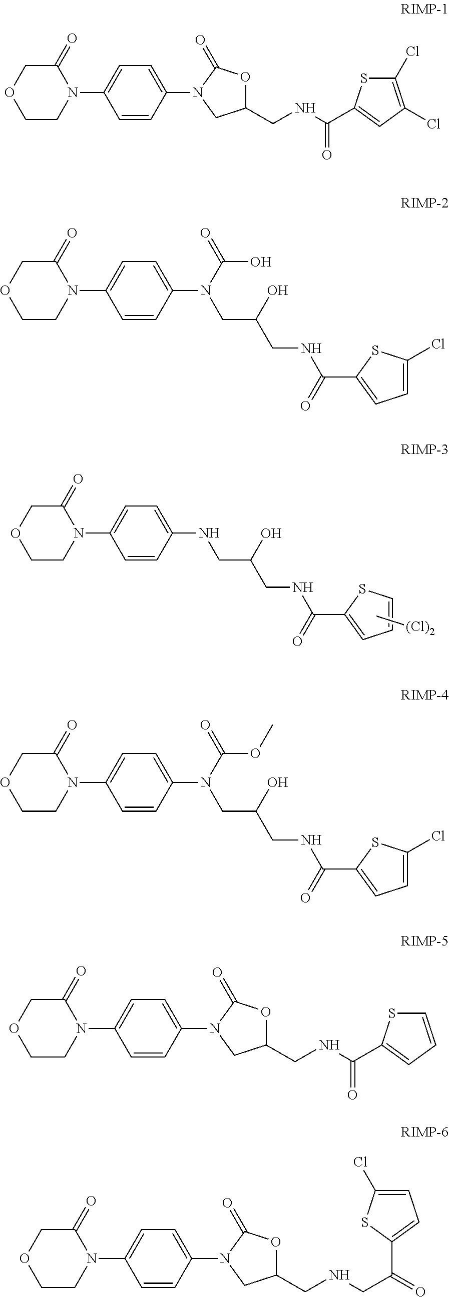

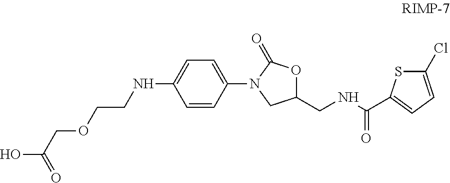 [SMILES: O=C(O)COCCNc1ccc(N2CC(CNC(=O)c3ccc(Cl)s3)OC2=O)cc1]

24. The process according to claim 3, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

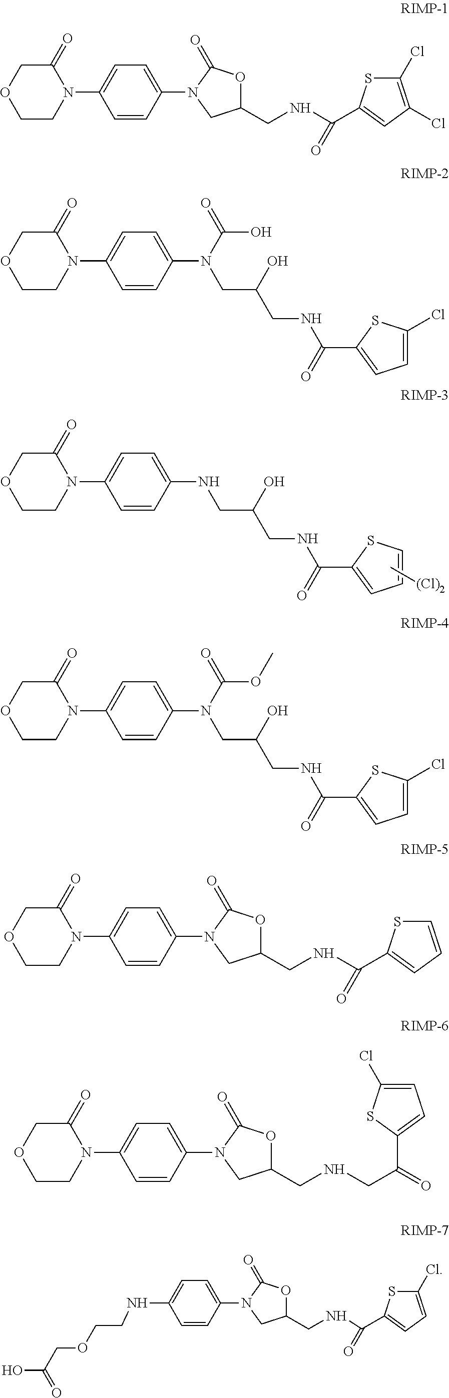

25. The process according to claim 4, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

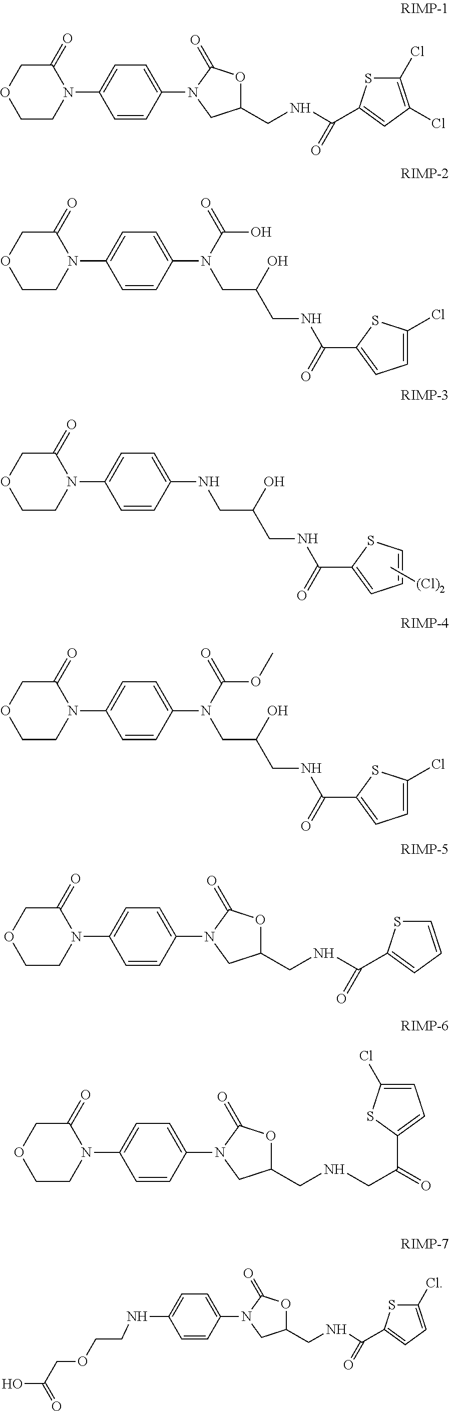

26. The process according to claim 5, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

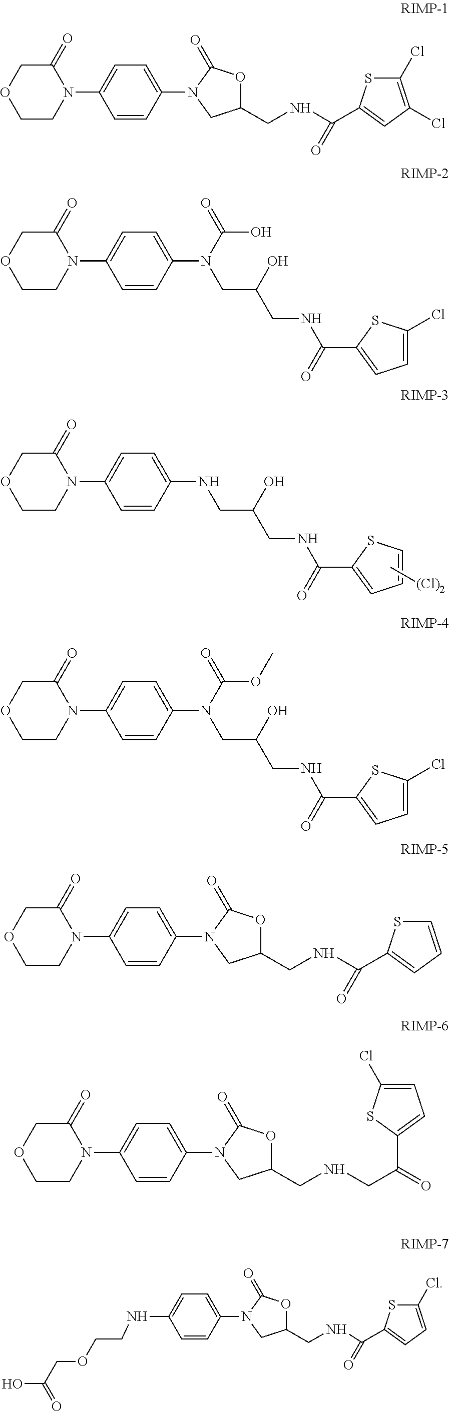

27. The process according to claim 6, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

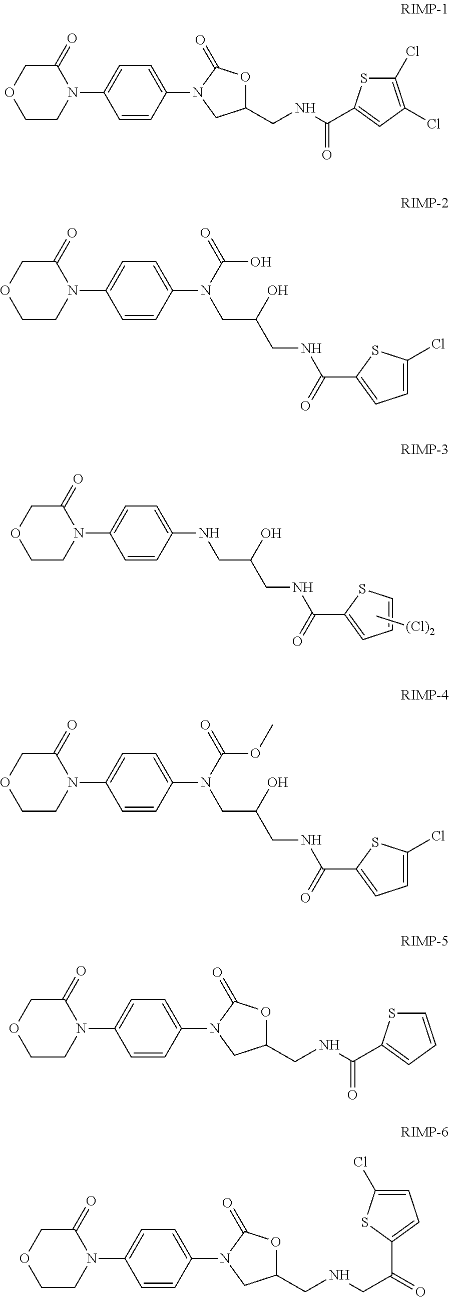

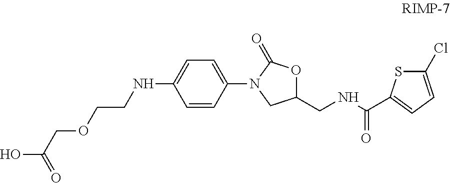 [SMILES: O=C(O)COCCNc1ccc(N2CC(CNC(=O)c3ccc(Cl)s3)OC2=O)cc1]

28. The process according to claim 7, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

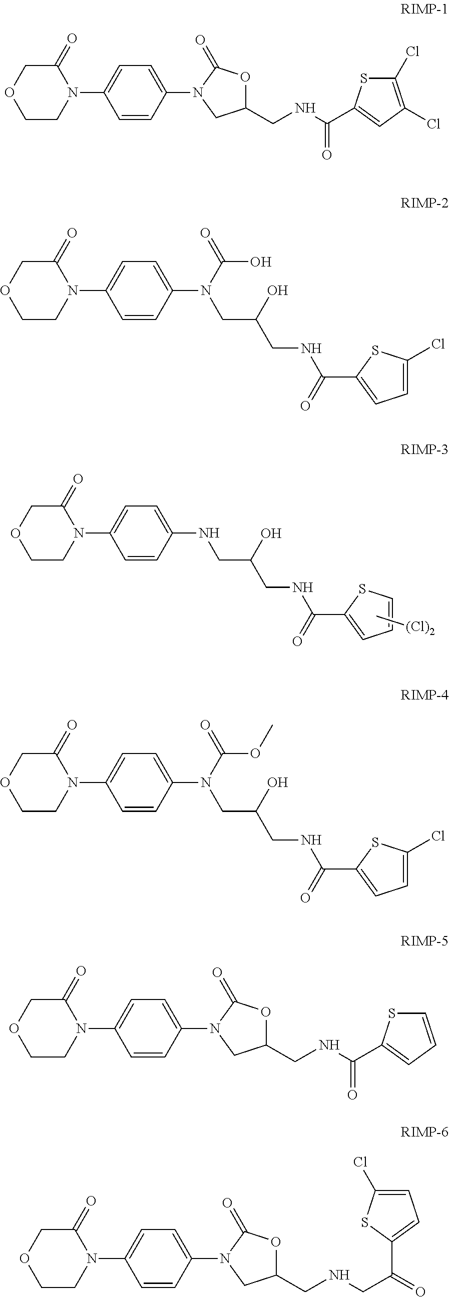

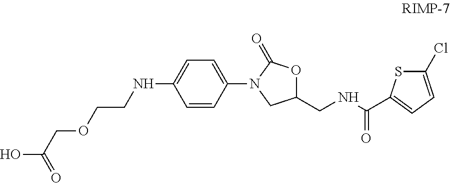 [SMILES: O=C(O)COCCNc1ccc(N2CC(CNC(=O)c3ccc(Cl)s3)OC2=O)cc1]

29. The process according to claim 8, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

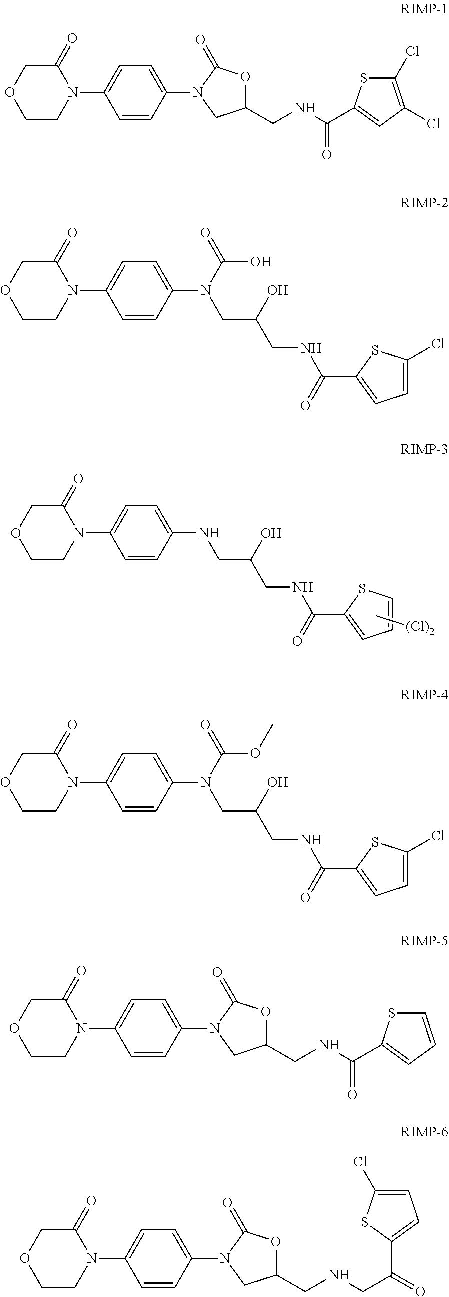

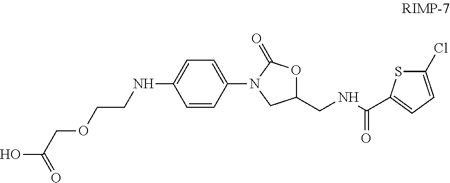 [SMILES: O=C(O)COCCNc1ccc(N2CC(CNC(=O)c3ccc(Cl)s3)OC2=O)cc1]

30. The process according to claim 9, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

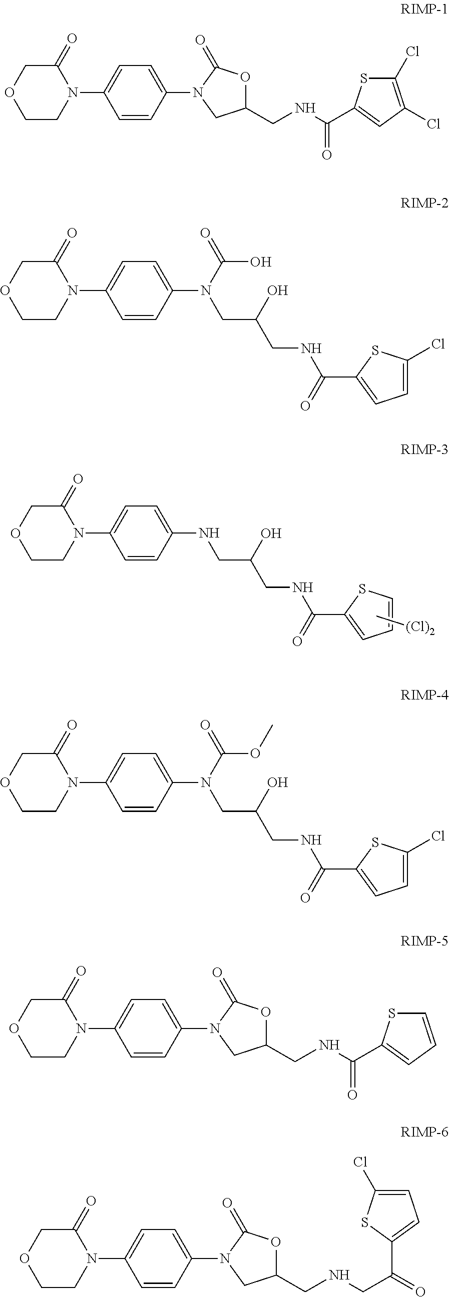

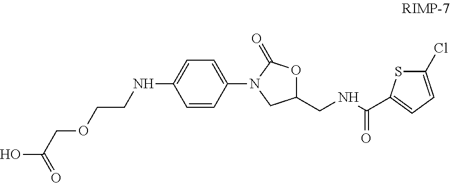 [SMILES: O=C(O)COCCNc1ccc(N2CC(CNC(=O)c3ccc(Cl)s3)OC2=O)cc1]

31. The process according to claim 10, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

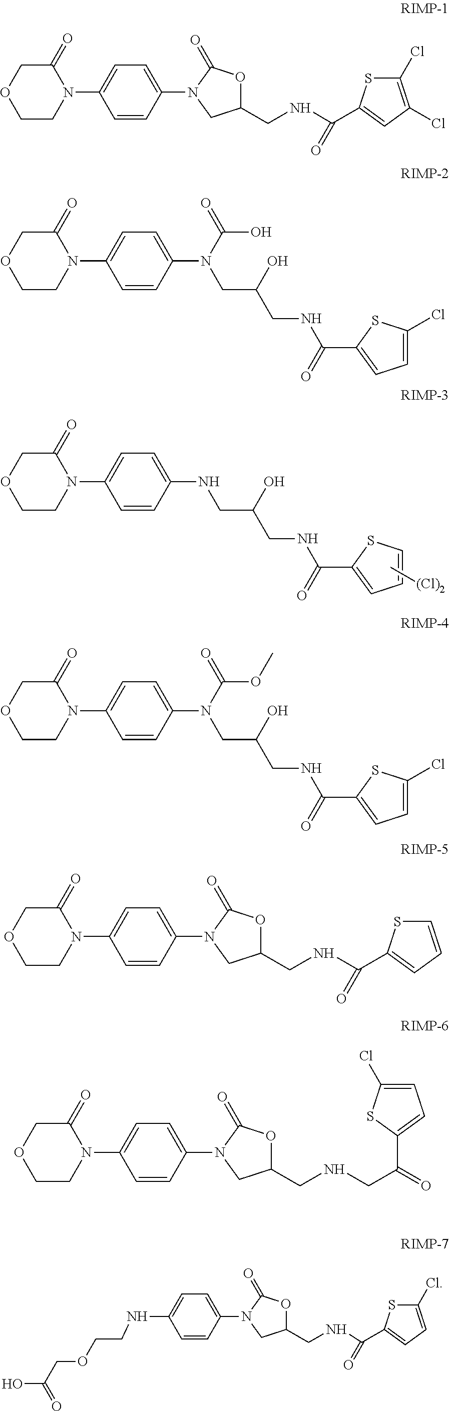

32. The process according to claim 11, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

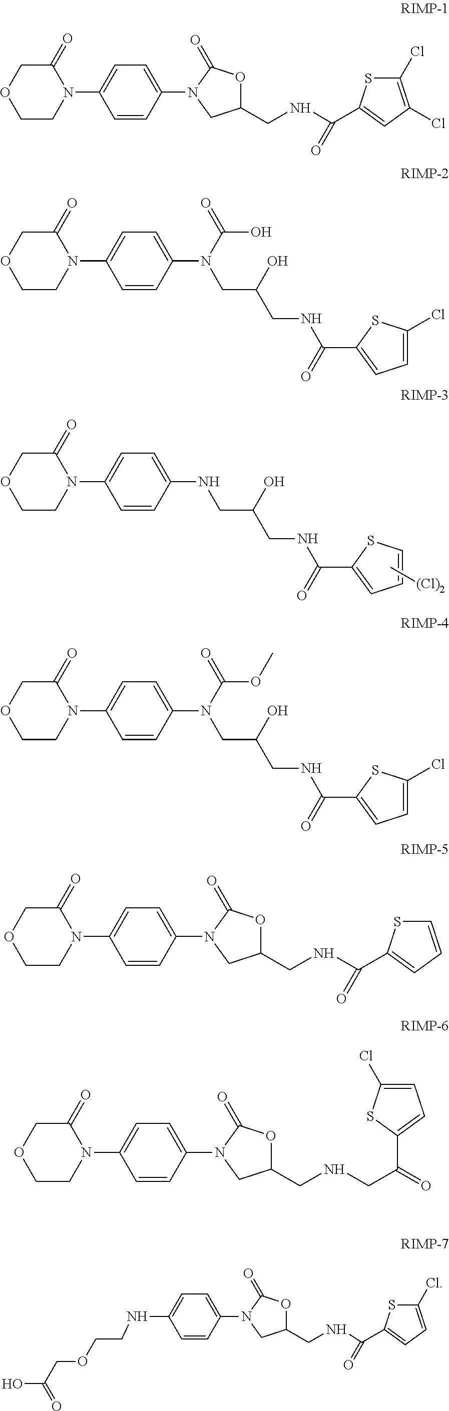

33. The process according to claim 12, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

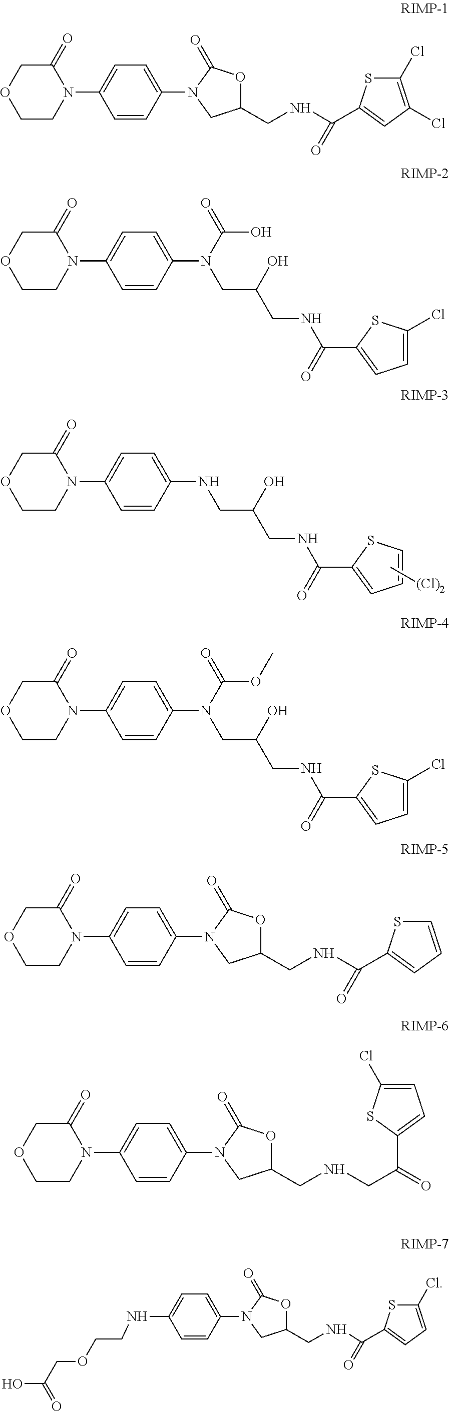

34. The process according to claim 13, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

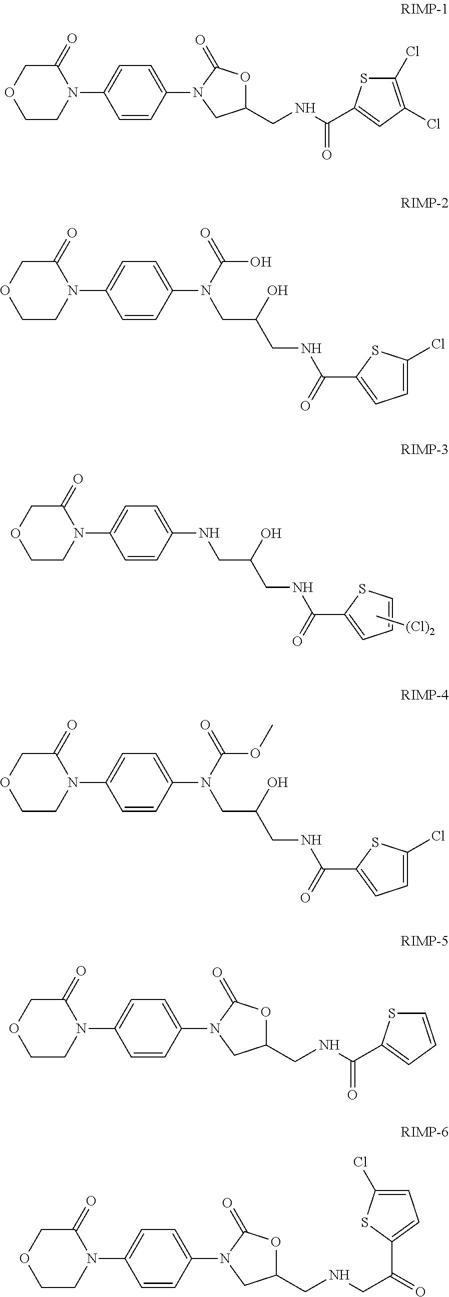

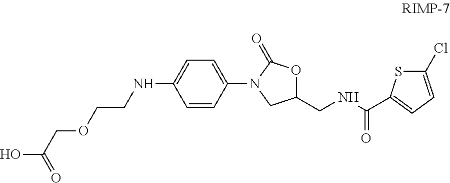 [SMILES: O=C(O)COCCNc1ccc(N2CC(CNC(=O)c3ccc(Cl)s3)OC2=O)cc1]

35. The process according to claim 14, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

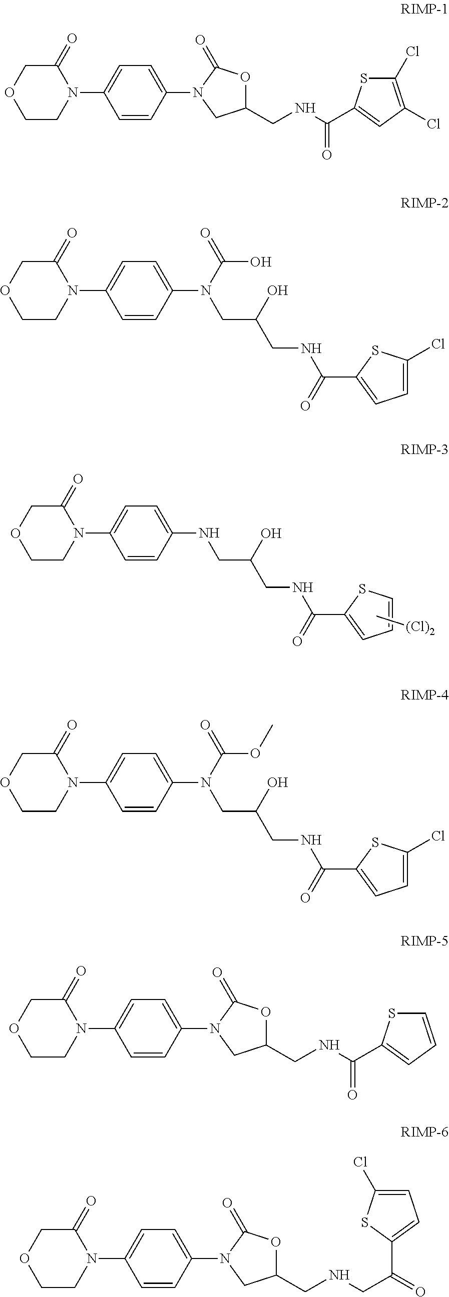

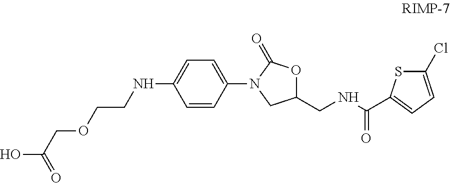 [SMILES: O=C(O)COCCNc1ccc(N2CC(CNC(=O)c3ccc(Cl)s3)OC2=O)cc1]

36. The process according to claim 15, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

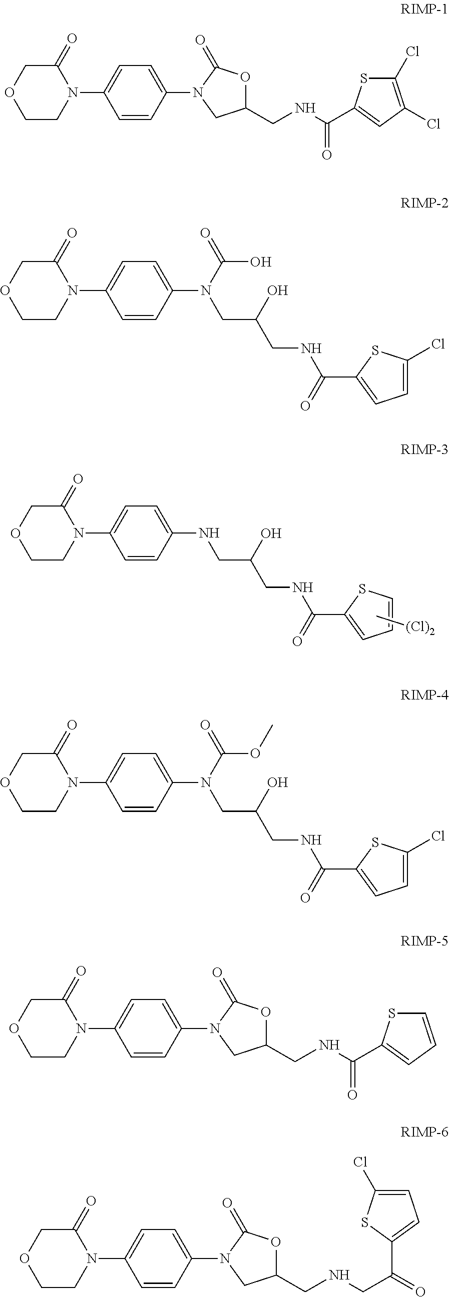

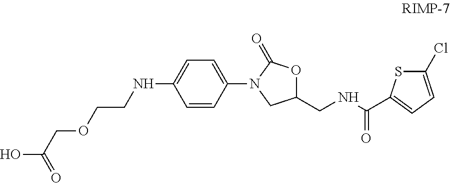 [SMILES: O=C(O)COCCNc1ccc(N2CC(CNC(=O)c3ccc(Cl)s3)OC2=O)cc1]

37. The process according to claim 16, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

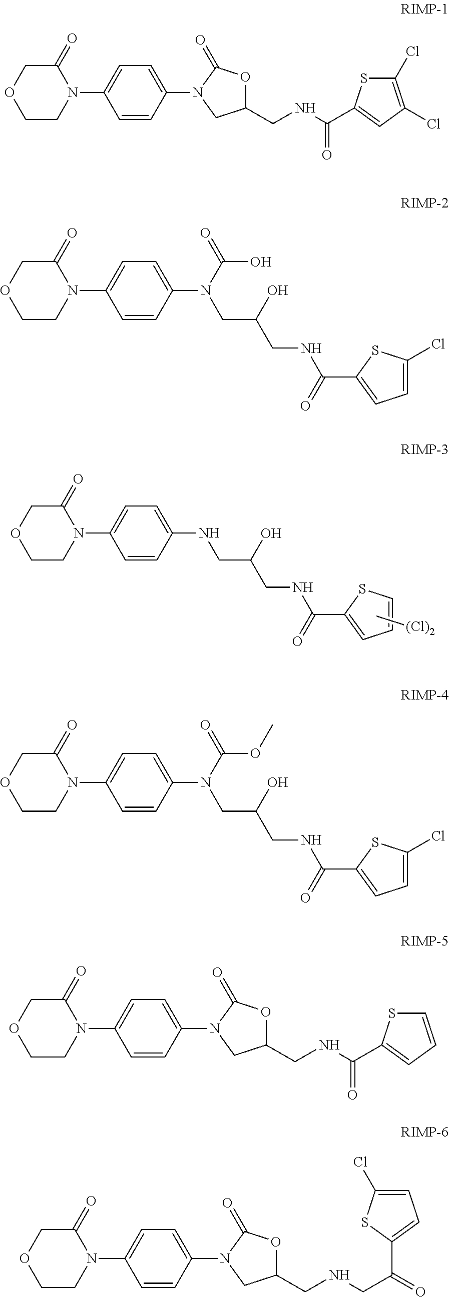

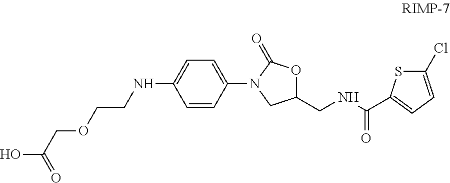 [SMILES: O=C(O)COCCNc1ccc(N2CC(CNC(=O)c3ccc(Cl)s3)OC2=O)cc1]

38. The process according to claim 17, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

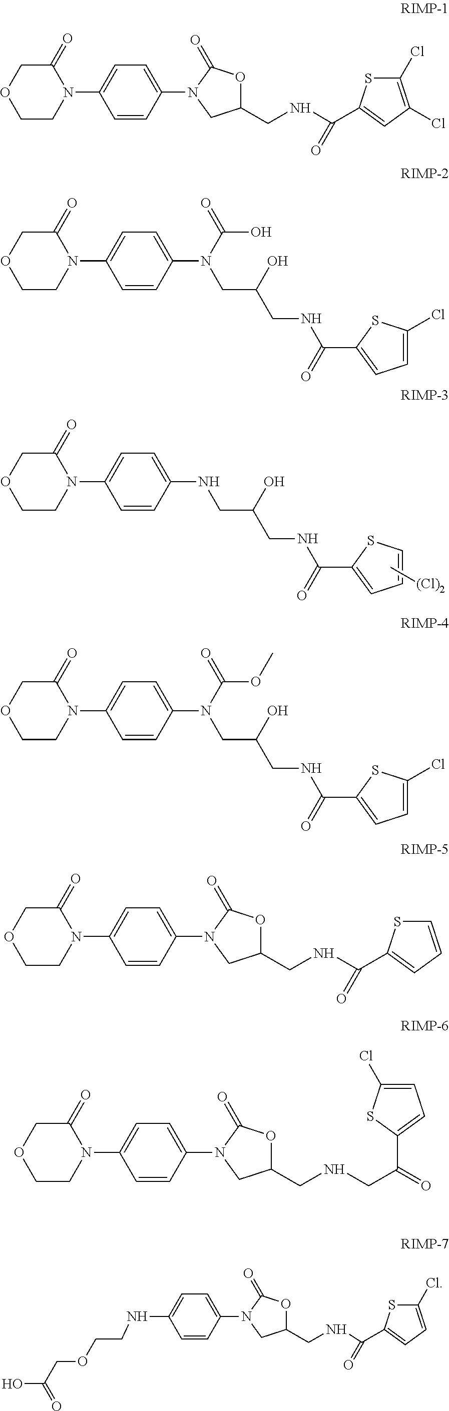

39. The process according to claim 18, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

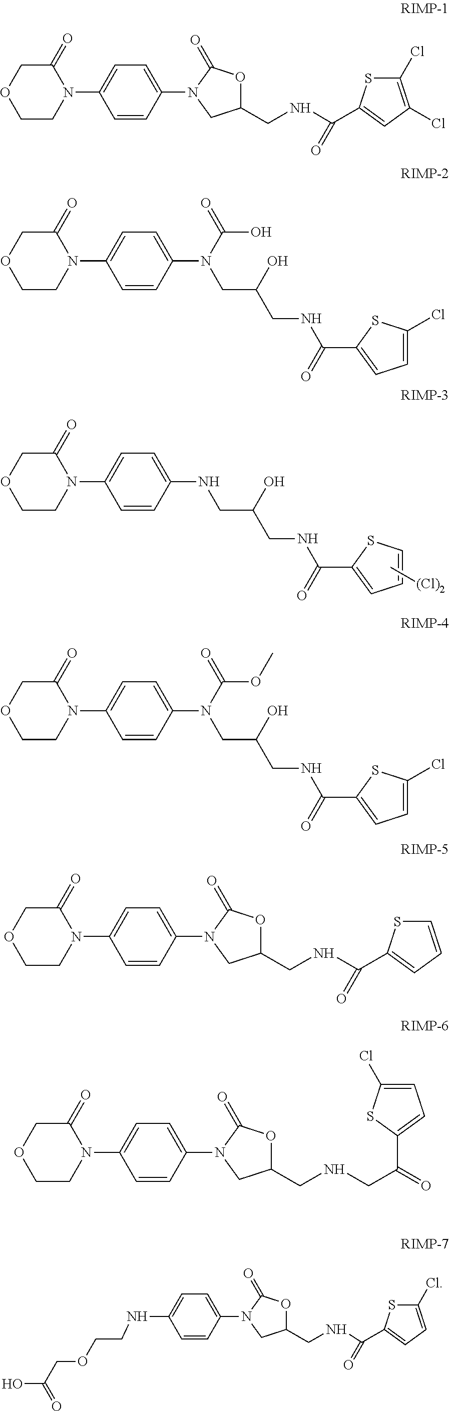

40. The process according to claim 19, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

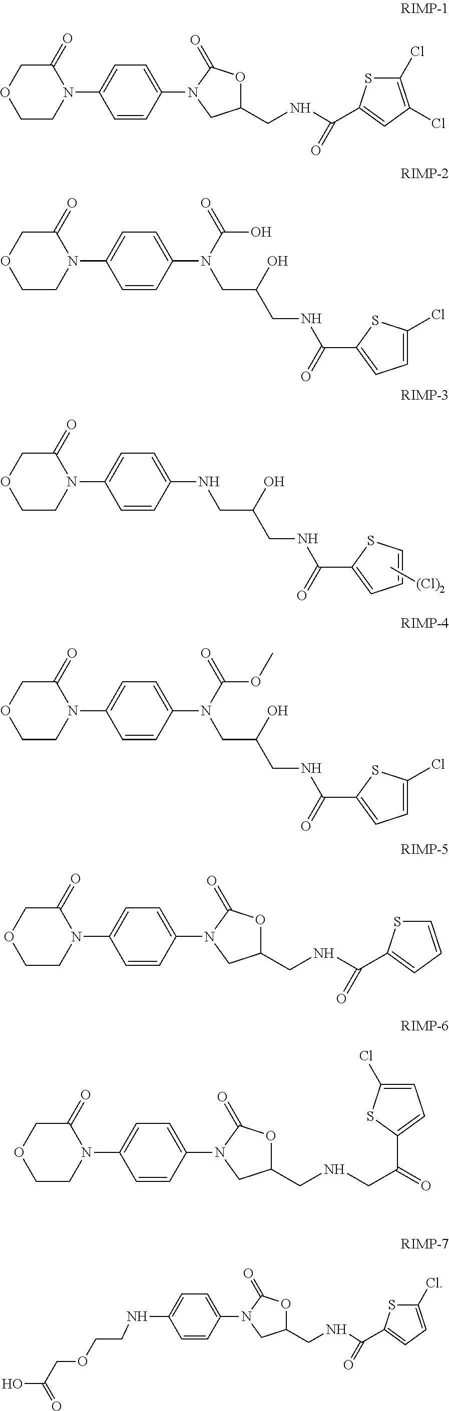

41. The process according to claim 20, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity.

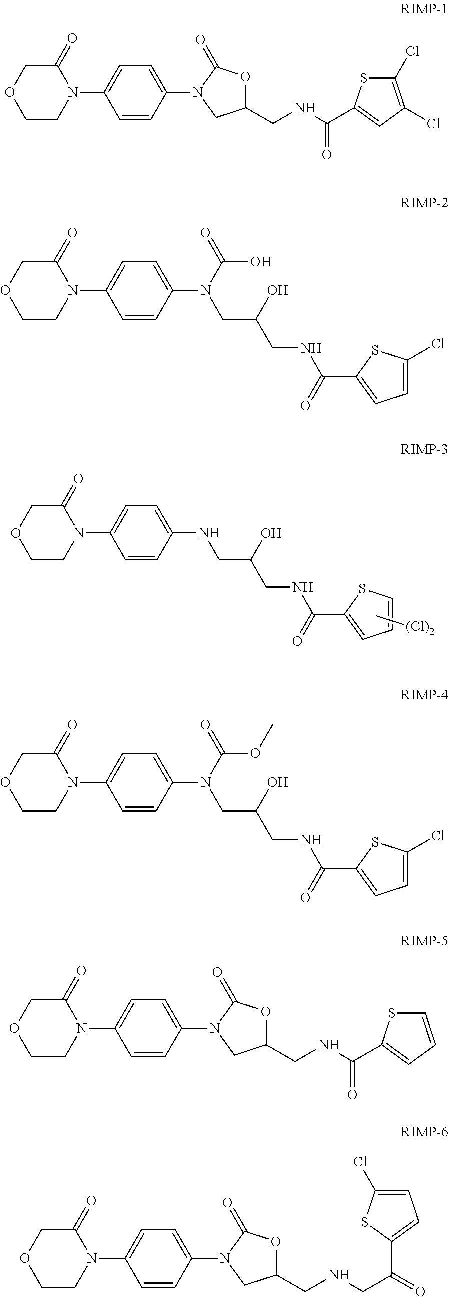

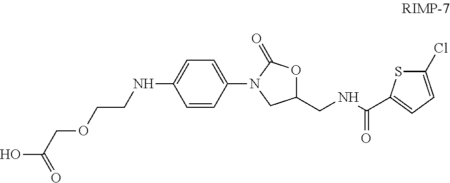 [SMILES: O=C(O)COCCNc1ccc(N2CC(CNC(=O)c3ccc(Cl)s3)OC2=O)cc1]

42. The process according to claim 21, wherein Rivaroxaban has less than about 0.2% of RIMP-1 impurity, has less than about 0.2% of RIMP-2 impurity, has less than about 0.2% RIMP-3 impurity, has less than about 0.2% RIMP-4 impurity, has less than about 0.2% RIMP-5 impurity, has less than about 0.2% of RIMP-6 impurity, has less than about 0.2% of RIMP-7 impurity. 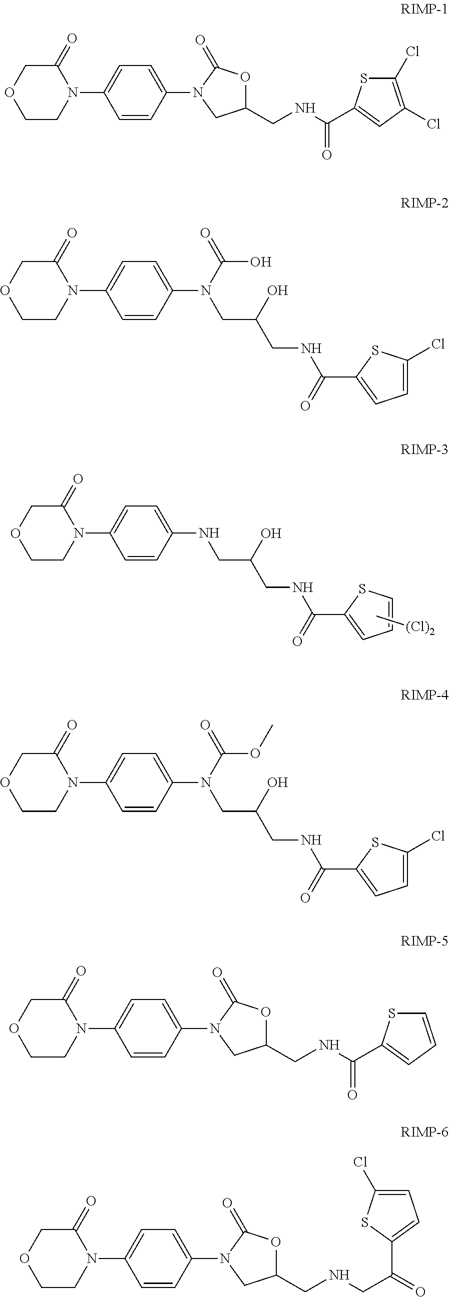 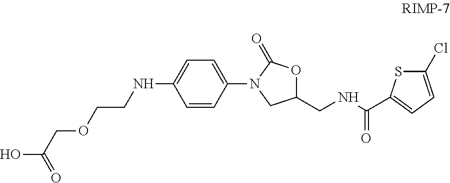 [SMILES: O=C(O)COCCNc1ccc(N2CC(CNC(=O)c3ccc(Cl)s3)OC2=O)cc1]
